(19) United States
(12) Patent Application Publication
(10) Pub. No.: US 2025/0090506 A1
(43) Pub. Date: Mar. 20, 2025

(54) COMBINATION THERAPY FOR THE TREATMENT OF MASTOCYTOSIS

(71) Applicant: Deciphera Pharmaceuticals, LLC, Waltham, MA (US)

(72) Inventors: Daniel L. Flynn, Waltham, MA (US); Bryan D. Smith, Waltham, MA (US); Anu Gupta, Lawrence, KS (US)

(21) Appl. No.: 18/815,054

(22) Filed: Aug. 26, 2024

Related U.S. Application Data
(63) Continuation of application No. 16/943,871, filed on Jul. 30, 2020, now Pat. No. 12,102,620.
Application No. 16/943,871 is a continuation of application No. PCT/US2019/016161, filed on Jan. 31, 2019.
(60) Provisional application No. 62/624,453, filed on Jan. 31, 2018.

(51) Int. Cl. A61K 31/4375; A61K 31/4184; A61K 31/4439; A61K 31/4523; A61K 31/506; A61K 31/519; A61K 45/06; A61P 43/00

(52) U.S. Cl. CPC A61K 31/4375; A61K 31/4184; A61K 31/4439; A61K 31/4523; A61K 31/506; A61K 31/519; A61K 45/06; A61P 43/00

(57) ABSTRACT

The present disclosure relates to the use of 1-[4-bromo-5-[1-ethyl-7-(methylamino)-2-oxo-1,2-dihydro-1,6-naphthyridin-3-yl]-2-fluorophenyl]-3-phenylurea or 1-(5-(7-amino-1-ethyl-2-oxo-1,2-dihydro-1,6-naphthyridin-3-yl)-4-bromo-2-fluorophenyl)-3-phenylurea, or a pharmaceutically acceptable salt thereof, in combination with a MAPKAP pathway inhibitor such as for example a RAS, RAF, MEK, or ERK inhibitor for the treatment of mastocytosis.

### CROSS-REFERENCE TO RELATED APPLICATIONS

This application is a continuation of U.S. Ser. No. 16/943,871 filed Jul. 30, 2020, which is a continuation of International Application Number PCT/US2019/016161 filed Jan. 31, 2019, which claims priority to U.S. Ser. No. 62/624,453 filed Jan. 31, 2018, the contents of which are incorporated herein by reference in their entirety.

### BACKGROUND

c-KIT (also known as KIT, CD117, and stem cell factor receptor) is a 145 kDa transmembrane tyrosine kinase protein that acts as a type-III receptor. The c-KIT proto-oncogene, located on chromosome 4q11-21, encodes the c-KIT receptor, whose ligand is the stem cell factor (SCF, steel factor, kit ligand, mast cell growth factor). The receptor has tyrosine-protein kinase activity, and binding of the ligand SCF leads to the autophosphorylation of c-KIT and its association with substrates such as phosphatidylinositol 3-kinase (PI3K). Tyrosine phosphorylation by protein tyrosine kinases is of particular importance in cellular signaling and can mediate signals for major cellular processes, such as proliferation, survival, differentiation, apoptosis, attachment, invasiveness and migration.

The receptor tyrosine kinase c-KIT gene is critical for mast cell growth, survival, differentiation and homeostasis. Activating mutations or overexpression of the c-KIT gene enhances the ability of the c-KIT receptor to initiate the intracellular pathways resulting in aberrant mast cell proliferation.

Mastocytosis is a rare disorder characterized by abnormal accumulations of mast cells (MCs) in the skin, bone marrow, and internal organs (e.g., liver, spleen, gastrointestinal tract and lymph nodes). Cases beginning during adulthood tend to be chronic and involve the bone marrow in addition to the skin, whereas, during childhood, the condition is often marked by skin manifestations with no internal organ involvement and can often resolve during puberty. In most adult patients, mastocytosis tends to be persistent, and may progress into a more advanced category in a minority of patients. Mastocytosis can be classified to a specific type depending on the patient's symptoms and overall presentation. Types of mastocytosis include cutaneous mastocytosis (e.g., maculopapular cutaneous mastocytosis, mastocytoma, and diffuse cutaneous mastocytosis) and systemic mastocytosis (e.g., indolent systemic mastocytosis (ISM), systemic smoldering mastocytosis (SSM), systemic mastocytosis with associated clonal hematological non-mast cell lineage disease (SM-AHN), aggressive systemic mastocytosis (ASM), mast cell leukemia (MCL) and mast cell sarcoma). In rare cases, outgrowth of aggressive neoplastic mast cells results in end organ failure where the patients have significantly reduced lifespan and require cytoreductive therapy. The pathologic accumulation of neoplastic mast cells caused by oncogenic mutations of c-KIT was found to be causative of systemic mastocytosis. The predominant activating KIT mutation is the aspartic acid to valine substitution at residue 816 (KIT D816V). Patients with systemic mastocytosis, whose mast cells frequently contain the activating D816V c-KIT mutation, may have indolent to aggressive diseases, and they may experience mast cell mediator release related symptoms. Indolent systemic mastocytosis with recurrent anaphylaxis or vascular collapse in the absence of skin lesions is a specific subtype indolent systemic mastocytosis, and this clonal MC activation disorder represents a significant fraction of all mast cell activation syndromes. The V560G KIT mutation is extremely rare in patients with systemic mastocytosis, and its biological and prognostic impact is unclear. Currently, most tyrosine kinase inhibitors have demonstrated only modest efficacy in advanced disease states and are accompanied by significant side effects. Additionally, some aggressive KIT mutations, including the KIT D816V mutation, are resistant to classical ATP competitive KIT inhibitors such as imatinib, sunitinib, sorafenib, and regorafenib. Midostaurin, an inhibitor of c-KIT D816V, was recently approved for the treatment of SM in 2017.

While the c-KIT D816V mutation is the primary c-KIT mutation reported as a driver of systemic mastocytosis (SM), secondary c-KIT mutations that confer resistance to certain c-KIT inhibitors (“secondary resistance c-KIT mutations”) have also been reported in mastocytosis patients, including, e.g., a Y269C, Y503_F504insAY, V560D, or K642E point mutation, an in-frame deletion or insertion, or a missense mutation in the c-KIT gene.

Activating mutations or overexpression of the c-KIT gene are linked to neoplastic mast cell proliferation. Given the complex function of c-KIT and the potential utility for c-KIT inhibitors in treating drug resistant systematic mastocytosis, there is a need for inhibitors and therapeutic treatments with advantageous therapeutic properties.

### SUMMARY

The present disclosure, in part, relates to the use of a c-KIT inhibitor, e.g., 1-[4-bromo-5-[1-ethyl-7-(methylamino)-2-oxo-1,2-dihydro-1,6-naphthyridin-3-yl]-2-fluorophenyl]-3-phenylurea (Compound A), and a MAPKAP pathway inhibitor, e.g., a MEK inhibitor such as trametinib, or an ERK inhibitor such as ulixertinib, or a RAF inhibitor such as LY3009120, for inducing the apoptosis of mastocytosis cells.

Also provided in the present disclosure are methods of treating mastocytosis in a patient in need thereof, comprising administering to the patient: an effective amount of a c-KIT inhibitor, e.g., 1-[4-bromo-5-[1-ethyl-7-(methylamino)-2-oxo-1,2-dihydro-1,6-naphthyridin-3-yl]-2-fluorophenyl]-3-phenylurea (Compound A as described herein); and an effective amount of an inhibitor of a mitogen-activated protein kinase (MEK inhibitor), and/or an effective amount of an extracellular signal regulated kinase inhibitor (ERK inhibitor).

For example, provided herein is a method of treating a systemic mastocytosis in a patient in need thereof, comprising administering to the patient: an effective amount of 1-[4-bromo-5-[1-ethyl-7-(methylamino)-2-oxo-1,2-dihydro-1,6-naphthyridin-3-yl]-2-fluorophenyl]-3-phenylurea, or a pharmaceutically acceptable salt thereof; and an effective amount of an inhibitor of a mitogen-activated protein kinase (MEK inhibitor) or ERK inhibitor.

Also contemplated in this disclosure is a method of treating mastocytosis in a patient in need thereof, comprising administering to the patient: an effective amount of a c-KIT inhibitor; and an effective amount of a RAF inhibitor.

### BRIEF DESCRIPTION OF THE DRAWINGS

FIG. 1A shows graphical representations of cell proliferation following the indicated drug treatment with Compound A as compared to vehicle control in HMC1.1 V560G (left panel) and HMC1.2 V560G/D816V (right panel) cell lines.

FIG. 1B shows graphical representations of cell proliferation following the indicated drug treatment with Compound B as compared to vehicle control in HMC1.1 V560G (left panel) and HMC1.2 V560G/D816V (right panel) cell lines.

FIG. 2A shows a graphical representation of caspase activity following the indicated various treatments with Compound A and trametinib in the HMC1.2 V560G/D816V cell line.

FIG. 2B provides a synergy matrix chart based on the combination index method for various treatments with Compound A and trametinib in the HMC1.2 V560G/D816V cell line.

FIG. 2C provides a Combination Index Plot for the combination of Compound A with the MEK inhibitor trametinib.

FIG. 3A shows a graphical representation of caspase activity following the indicated various treatments with Compound B and trametinib in the HMC1.2 V560G/D816V cell line.

FIG. 3B provides a synergy matrix chart based on the combination index method for various treatments with Compound B and trametinib in the HMC1.2 V560G/D816V cell line.

FIG. 3C provides a Combination Index Plot for the combination of Compound B with the MEK inhibitor trametinib.

FIG. 4A shows a graphical representation of caspase activity following the indicated various treatments with Compound A and binimetinib in the HMC1.2 V560G/D816V cell line.

FIG. 4B provides a synergy matrix chart based on the combination index method for various treatments with Compound A and binimetinib in the HMC1.2 V560G/D816V cell line.

FIG. 4C provides a Combination Index Plot for the combination of Compound A with the MEK inhibitor binimetinib.

FIG. 5A shows a graphical representation of caspase activity following the indicated various treatments with Compound B and binimetinib in the HMC1.2 V560G/D816V cell line.

FIG. 5B provides a synergy matrix chart based on the combination index method for various treatments with Compound B and binimetinib in the HMC1.2 V560G/D816V cell line.

FIG. 5C provides a Combination Index Plot for the combination of Compound B with the MEK inhibitor binimetinib.

FIG. 6A shows a graphical representation of caspase activity following the indicated various treatments with Compound A and cobimetinib in the HMC1.2 V560G/D816V cell line.

FIG. 6B provides a synergy matrix chart based on the combination index method for various treatments with Compound A and cobimetinib in the HMC1.2 V560G/D816V cell line.

FIG. 6C provides a Combination Index Plot for the combination of Compound A with the MEK inhibitor cobimetinib.

FIG. 7A shows a graphical representation of caspase activity following the indicated various treatments with Compound B and cobimetinib in the HMC1.2 V560G/D816V cell line.

FIG. 7B provides a synergy matrix chart based on the combination index method for various treatments with Compound B and cobimetinib in the HMC1.2 V560G/D816V cell line.

FIG. 7C provides a Combination Index Plot for the combination of Compound B with the MEK inhibitor cobimetinib.

FIG. 8A shows a graphical representation of caspase activity following the indicated various treatments with Compound A and the ERK inhibitor ulixertinib in the HMC1.2 V560G/D816V cell line.

FIG. 8B provides a synergy matrix chart based on the combination index method for various treatments with Compound A and the ERK inhibitor ulixertinib in the HMC1.2 V560G/D816V cell line.

FIG. 8C provides a Combination Index Plot for the combination of Compound A with the ERK inhibitor ulixertinib.

FIG. 9A shows the inhibition of colony outgrowth from treatment with single agent Compound A, single agent trametinib, and the combination of Compound A with the MEK inhibitor trametinib in an HMC1.2 V560G/D816V cells.

FIG. 9B shows a graphical representation of inhibition of colony outgrowth from treatment with single agent Compound A, single agent trametinib, and the combination of Compound A with the MEK inhibitor trametinib in HMC1.2 V560G/D816V cells. Arrows indicate no colony outgrowth.

FIG. 10A shows the inhibition of colony outgrowth from treatment with single agent Compound B, single agent trametinib, and the combination of Compound B with the MEK inhibitor trametinib in an HMC1.2 V560G/D816V cells.

FIG. 10B shows a graphical representation of inhibition of colony outgrowth from treatment with single agent Compound B, single agent trametinib, and the combination of Compound B with the MEK inhibitor trametinib in HMC1.2 V560G/D816V cells. Arrows indicate no colony outgrowth.

FIG. 11A shows the inhibition of colony outgrowth from treatment with single agent Compound A, single agent binimetinib, and the combination of Compound A with the MEK inhibitor binimetinib in an HMC1.2 V560G/D816V cells.

FIG. 11B shows a graphical representation of inhibition of colony outgrowth from treatment with single agent Compound A, single agent binimetinib, and the combination of Compound A with the MEK inhibitor binimetinib in HMC1.2 V560G/D816V cells. Arrows indicate no colony outgrowth.

FIG. 12A shows the inhibition of colony outgrowth from treatment with single agent Compound B, single agent binimetinib, and the combination of Compound B with the MEK inhibitor binimetinib in an HMC1.2 V560G/D816V cells.

FIG. 12B shows a graphical representation of inhibition of colony outgrowth from treatment with single agent Compound B, single agent binimetinib, and the combination of Compound B with the MEK inhibitor binimetinib in HMC1.2 V560G/D816V cells. Arrows indicate no colony outgrowth.

FIG. 13A shows the inhibition of colony outgrowth from treatment with single agent Compound A, single agent cobimetinib, and the combination of Compound A with the MEK inhibitor cobiimetinib in an HMC1.2 V560G/D816V cells.

FIG. 13B shows a graphical representation of inhibition of colony outgrowth from treatment with single agent Compound A, single agent cobimetinib, and the combination of Compound A with the MEK inhibitor cobimetinib in HMC1.2 V560G/D816V cells.

FIG. 14A shows the inhibition of colony outgrowth from treatment with single agent Compound B, single agent cobimetinib, and the combination of Compound B with the MEK inhibitor cobimetinib in an HMC1.2 V560G/D816V cells.

FIG. 14B shows a graphical representation of inhibition of colony outgrowth from treatment with single agent Compound B, single agent cobimetinib, and the combination of Compound B with the MEK inhibitor cobimetinib in HMC1.2 V560G/D816V cells.

FIG. 15A shows a graphical representations of caspase activity following various treatments with Compound A and trametinib at 24 hours in empty vector (EV, left panel)) or N-ras G12D (right panel) transfected HMC1.2 V560G/D816V cells.

FIG. 15B shows a graphical representations of caspase activity following various treatments with Compound A and trametinib at 48 hours in empty vector (left panel) or N-ras G12D (right panel) transfected HMC1.2 V560G/D816V cells.

FIG. 16A shows a graphical representations of caspase activity following various treatments with Compound A and cobimetinib at 24 hours in empty vector (EV, left panel) or N-ras G12D (right panel) transfected HMC1.2 V560G/D816V cells.

FIG. 16B shows a graphical representations of caspase activity following various treatments with Compound A and cobimetinib at 48 hours in empty vector (left panel) or N-ras G12D (right panel) transfected HMC1.2 V560G/D816V cells.

FIG. 17A shows the inhibition of colony outgrowth from treatment with single agent Compound A, single agent trametinib, and the combination of Compound A with the MEK inhibitor trametinib in an empty vector (EV) transfected HMC1.2 V560G/D816V cells.

FIG. 17B shows a graphical representation of inhibition of colony outgrowth from treatment with single agent Compound A, single agent trametinib, and the combination of Compound A with the MEK inhibitor trametinib in an empty vector (EV) transfected HMC1.2 V560G/D816V cells. Arrows indicate no colony outgrowth.

FIG. 17C shows the inhibition of colony outgrowth from treatment with single agent Compound A, single agent trametinib, and the combination of Compound A with the MEK inhibitor trametinib in an N-ras G12D transfected HMC1.2 V560G/D816V cells.

FIG. 17D shows a graphical representation of inhibition of colony outgrowth from treatment with single agent Compound A, single agent trametinib, and the comination of Compound 1 with the MEK inhibitor trametinib in an N-ras G12D transfected HMC1.2 V560G/D816V cells. Arrows indicate no colony outgrowth.

FIG. 18A shows the inhibition of colony outgrowth from treatment with single agent Compound A, single agent cobimetinib, and the combination of Compound A with the MEK inhibitor cobimetinib in an empty vector (EV) transfected HMC1.2 V560G/D816V cells.

FIG. 18B shows a graphical representation of inhibition of colony outgrowth from treatment with single agent Compound A, single agent cobimetinib, and the combination of Compound A with the MEK inhibitor cobimetinib in an empty vector (EV) transfected HMC1.2 V560G/D816V cells. Arrows indicate no colony outgrowth.

FIG. 18C shows the inhibition of colony outgrowth from treatment with single agent Compound A, single agent cobimetinib, and the combination of Compound A with the MEK inhibitor cobimetinib in an N-ras G12D transfected HMC1.2 V560G/D816V cells.

FIG. 18D shows a graphical representation of inhibition of colony outgrowth from treatment with single agent Compound A, single agent cobimetinib, and the combination of Compound A with the MEK inhibitor cobimetinib in an N-ras G12D transfected HMC1.2 V560G/D816V cells. Arrows indicate no colony outgrowth.

### DETAILED DESCRIPTION

It is found that the combination of a c-KIT inhibitor, e.g., 1-[4-bromo-5-[1-ethyl-7-(methylamino)-2-oxo-1,2-dihydro-1,6-naphthyridin-3-yl]-2-fluorophenyl]-3-phenylurea (Compound A), and a MAPKAP pathway inhibitor, e.g., the MEK inhibitor trametinib, or the ERK inhibitor ulixertinib, or the RAF inhibitor LY3009120 unexpectedly synergizes to induce apoptosis of mastocytosis cells, as demonstrated in the accompanying Examples. In addition, the combination therapy methods disclosed herein are cytocidal as opposed to being merely cytostatic.

Without wishing to be bound to any particular theory, it is believed that many c-KIT inhibitors only inhibit certain mutant forms of c-KIT and do not effectively inhibit the c-KIT D816V mutation that is causative of SM. The present disclosure provides methods of treating c-KIT-mediated mastocytosis, by inhibiting both c-KIT and MEK, using a c-KIT inhibitor in combination with a MAPKAP pathway inhibitor. In particular embodiments, the c-KIT inhibitor is disclosed herein as Compound A or a pharmaceutically acceptable salt thereof, Compound B or a pharmaceutically acceptable salt thereof, midostaurin or a pharmaceutically acceptable salt thereof, BLU-285 or a pharmaceutically acceptable salt thereof, PLX9486 or a pharmaceutically acceptable salt thereof, or crenolanib or a pharmaceutically acceptable salt thereof. Surprisingly, the c-KIT inhibitor, e.g., Compound A and Compound B (and pharmaceutically acceptable salts thereof), synergizes with the MEK, ERK,or RAF, inhibitor to inhibit proliferation and induce apoptosis of mastocytosis cells.

Accordingly, in certain embodiments, the present disclosure provides methods for inducing inducing cytocidal mast cell killing, inducing apoptosis of mast cells, inhibiting growth or proliferation of mast cells, inhibiting mast cell mediator release, reducing the amount of accumulated mast cells in a tissue or organ, reducing the volume of a mastocytosis-related tumor, such as a mast cell leukemia or mast cell sarcoma, and/or inhibiting mast cell regrowth, by contacting mast cells, e.g., mast cells comprising a c-KIT mutation, with a c-KIT inhibitor and a MAPKAP pathway inhibitor. In various embodiments, the mast cells are contacted in vitro, in vivo, or ex vivo. In particular embodiments, the c-KIT inhibitor is disclosed herein as Compound A or a pharmaceutically acceptable salt thereof, Compound B or a pharmaceutically acceptable salt thereof, midostaurin or a pharmaceutically acceptable salt thereof, BLU-285 or a pharmaceutically acceptable salt thereof, PLX9486 or a pharmaceutically acceptable salt thereof, or crenolanib or a pharmaceutically acceptable salt thereof; and the MEK inhibitor disclosed herein as trametinib, cobimetinib, selumetinib, or binimetinib; ERK inhibitors including but not limited to ulixertinib, SCH772984, LY3214996, ravoxertinib, and VX-11e, and RAF inhibitors including but not limited to LY3009120, vemurafenib, or dabrafenib.

In certain embodiments, the present disclosure includes methods for treating a subject having a mastocytosis, a mast cell leukemia, or an acute myeloid leukemia, comprising administering to the subject an effective amount of: (i) a KIT inhibitor, e.g., 1-[4-bromo-5-[1-ethyl-7-(methylamino)-2-oxo-1,2-dihydro-1,6-naphthyridin-3-yl]-2-fluorophenyl]-3-phenylurea or a pharmaceutically acceptable salt thereof, or 1-(5-(7-amino-1-ethyl-2-oxo-1,2-dihydro-1,6-naphthyridin-3-yl)-4-bromo-2-fluorophenyl)-3-phenylurea or a pharmaceutically acceptable salt thereof; and (ii) a MAPKAP pathway inhibitor, e.g., trametinib, binimetinib, ulixertinib. In particular embodiments of any of the methods disclosed herein, the mastocytosis is a systemic mastocytosis resulting from a D816V mutation of the c-KIT gene in mast cells.

### Definitions

“Compounds A and B” as used herein refer to 1-[4-bromo-5-[1-ethyl-7-(methylamino)-2-oxo-1,2-dihydro-1,6-naphthyridin-3-yl]-2-fluorophenyl]-3-phenylurea and 1-(5-(7-amino-1-ethyl-2-oxo-1,2-dihydro-1,6-naphthyridin-3-yl)-4-bromo-2-fluorophenyl)-3-phenylurea, respectively. Pharmaceutically acceptable salts, tautomers, hydrates, and solvates, of Compounds A and B are also contemplated in this disclosure. The structures of Compounds A and B are represented below:

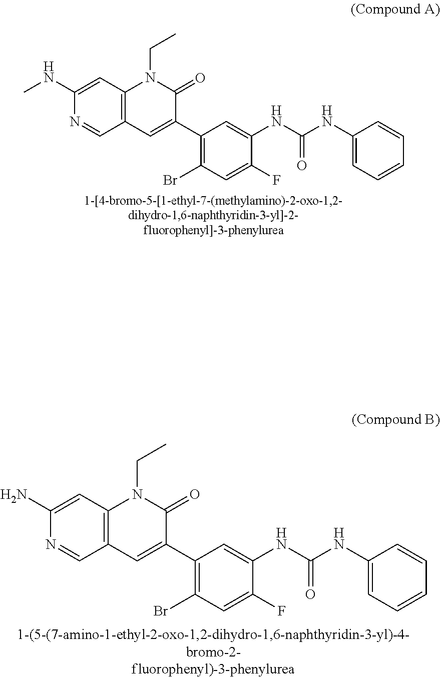 [SMILES: CCn1c(=O)c(-c2cc(NC(=O)Nc3ccccc3)c(F)cc2Br)cc2cnc(NC)cc21 ; CCn1c(=O)c(-c2cc(NC(=O)Nc3ccccc3)c(F)cc2Br)cc2cnc(N)cc21]

Methods of making Compound A and Compound B are disclosed in U.S. Pat. No. 8,461,179B1, the contents of which are incorporated herein by reference.

Illustrative methods and materials are described herein. In the specification and the appended claims, the singular forms also include the plural unless the context clearly dictates otherwise. Unless defined otherwise, all technical and scientific terms used herein have the same meaning as commonly understood by one of ordinary skill in the art.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications and this disclosure. For convenience, certain terms employed in the specification, examples and claims are collected here. Unless defined otherwise, all technical and scientific terms used in this disclosure have the same meanings as commonly understood by one of ordinary skill in the art to which this disclosure belongs. The initial definition provided for a group or term provided in this disclosure applies to that group or term throughout the present disclosure individually or as part of another group, unless otherwise indicated.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes acid addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

A “pharmaceutical composition” refers to a formulation of a compound described herein (e.g., Compound A or a pharmaceutically acceptable salt thereof) and a medium generally accepted in the art for the delivery of the biologically active compound to mammals, e.g., humans. Such a medium includes all pharmaceutically acceptable carriers, diluents or excipients therefor.

A “MAPKAP pathway inhibitor” is an inhibitor of the MAP kinase signaling pathway. Inhibitors of this pathway include RAS inhibitors, RAF inhibitors (e.g. vemurafenib, dabrafenib, LY3009120), MEK inhibitors (e.g. trametinib, binimetinib, cobimetinib), and ERK inhibitors (e.g. ulixertinib).

Subjects or patients “in need of treatment” with a combination therapy of the present disclosure, e.g., a c-KIT inhibitor in combination with a MAPKAP pathway inhibitor, include subjects with diseases and/or conditions that can be treated with a combination disclosed herein to achieve a beneficial therapeutic result, e.g., a mastocytosis, mast cell leukemia, or acute myeloid leukemia. A beneficial outcome in the treatment of mastocytosis may include a complete response, a partial response, a clinical improvement, or stable disease as defined by IWG-MRT-ECNM criteria (Gotlib et al, Blood 2013; 121:2393-401). In certain embodiments, a subject in need of treatment is suffering from a cutaneous mastocytosis (e.g., maculopapular cutaneous mastocytosis, mastocytoma, and diffuse cutaneous mastocytosis) or a systemic mastocytosis (e.g., indolent systemic mastocytosis, systemic smoldering mastocytosis, systemic mastocytosis with associated clonal hematological non-mast cell lineage disease, aggressive systemic mastocytosis, mast cell leukemia and mast cell sarcoma). In particular embodiments, subjects are mammals, e.g., humans or other mammals.

The term “effective amount” when used in connection with a compound or other therapeutic agent disclosed herein, refers to an amount of the therapeutic agent, e.g., Compound A or a MAPKAP pathway inhibitor, alone or in combination, that is useful to treat or prevent a disease or disorder. The effective amount of therapeutic agents used in a combination therapy is the amount of each of the therapeutic agents that is useful for treating or preventing a disease or disorder when used in the combination therapy, even if the amount of one or both of the therapeutic agents, in the absence of the other therapeutic agent, is ineffective to treat or prevent the disease or disorder. In certain embodiments, an effective amount is a quantity that results in inducing cytocidal mast cell killing, inducing apoptosis of mast cells, reducing the amount of accumulated mast cells in a tissue or organ, reducing mastocytosis symptoms, inhibiting mast cell mediator release, inhibiting the growth of mast cells, and/or inducing mastocytosis regression wherein mast cells harbor activating mutations in c-KIT kinase including the activating c-KIT D816V mutation. The “effective amount” can vary depending upon the mode of administration, specific locus of the disease or disorder, and the age, body weight, and general health of the subject. The amount of the compounds administered will depend on the degree, severity, and type of the disease or condition, the amount of therapy desired, and the release characteristics of the pharmaceutical formulation(s). It will also depend on the subject's health, size, weight, age, sex and tolerance to drugs. Typically, the compounds are administered for a sufficient period of time to achieve the desired therapeutic effect.

The terms “treatment,” “treat,” and “treating,” are meant to include the full spectrum of intervention in patients being treated, e.g., patients with a mastocytosis or acute myeloid leukemia (AML). Treating can be curing, improving, or at least partially ameliorating the disorder. In particular embodiments, treatment is performed with the intention to induce cytocidal mast cell killing, induce apoptosis of mast cells, reduce the amount of accumulated mast cells in a tissue or organ, reduce mastocytosis symptoms, inhibit mast cell mediator release, inhibit the growth of mast cells, and/or induce mastocytosis regression in the subject being treated. In certain embodiments, treatment with a combination therapy disclosed herein alleviates, slows or reverses one or more of the mastocytosis symptoms and/or induces regression of the mastocytosis, even if the mastocytosis is not actually eliminated. In some embodiments, treatment includes eliminating the disease or disorder, e.g., mastocytosis or AML, entirely. In other embodiments, treatment results in a complete response, a partial response, a clinical improvement, or stable disease as defined by IWG-MRT-ECNM criteria (Gotlib et al., Blood 2013; 121:2393-401).

“Mast cells” as used herein, include mast cells (also called mastocytes) and CD34+ mast cell precursor cells.

“Neoplasm” as used herein refers to an abnormal tissue that grows by cellular proliferation more rapidly than normal. Neoplasms show partial or complete lack of structural organization and functional coordination with the normal tissue, and usually form a distinct mass of tissue that may be either benign (benign tumor) or malignant (cancer).

“Tumor” as used herein refers to a mass. This is a term that may refer to benign (generally harmless) or malignant (cancerous) growths. Malignant growth can originate from a solid organ or the bone marrow. The latter is often referred to as liquid tumors. “Tumors” encompass mast cell leukemias and mast cell sarcomas, collectively referred to herein as “mastocytosis tumors.”

In certain embodiments, the therapeutic effect of treating a mastocytosis according to the methods disclosed herein may be measured using standard response criteria known in the art. For example, “response criteria for complete remission (CR), partial remission (PR), clinical improvement (CI) or stable disease (SD)” used to quantitate the effects of therapy on aggressive systemic mastocytosis, mast cell leukemia, and systemic mastocytosis associated with a myeloid neoplasm may be any of those criteria defined by the IWG-MRT-ECNM (Gotlib et al, Blood 2013; 121: 2393-401). For example, Complete remission (CR) requires all 4 criteria and response duration must be≥12 wk, no presence of compact neoplastic mast cell aggregates in the BM or other biopsied extracutaneous organ. Serum tryptase level<20 ng/mL†, peripheral blood count remission defined as ANC≥1×10⁹/L with normal differential, Hb level≥11 g/dL, and platelet count≥100×10⁹/L, and complete resolution of palpable hepatosplenomegaly and all biopsy-proven or suspected SM-related organ damage (CI findings); Partial remission (PR)*requires all 3 criteria and response duration must be≥12 wk, in the absence of both CR and progressive disease (PD) with: reduction by≥50% in neoplastic MCs in the marrow and/or or other extracutaneous organ at biopsy demonstrating eligible SM-related organ damage, reduction of serum tryptase level by≥50%; and resolution of 1 or more biopsy-proven or suspected SM-related organ damage (CI finding(s)); clinical improvement (CI) is where response duration must be≥12 wk, requires 1 or more of the nonhematologic and/or hematologic response criteria to be fulfilled (see Table 3) in the absence of both CR/PR, and assignment or progressive disease (PD). Stable disease (SD) is not meeting criteria for CR, PR, CI, or PD.

Guidelines for adjudicating response include (1) Only disease-related≥grade 2 organ damage is evaluable as a primary endpoint in clinical trials. (2) Response adjudications of CR, PR, SD, PD, and loss or response (LOR) should only be applied to these≥grade 2 organ damage findings in the context of trials. (3) Disease status at the time of patient removal from the study singularly relates to the updated status of initial≥grade 2 organ damage finding(s). (4) Exclusion of drug-related toxicity and/or other clinical issues (e.g., gastrointestinal tract bleeding in the case of worsening anemia/transfusion-dependence) should be undertaken before assigning the designation PD or LOR in a patient with worsening of baseline≥grade 2 organ damage.

In certain embodiments, the therapeutic effect of treating an AML according to the methods disclosed herein may be measured using standard response criteria known in the art. For example, “response criteria for the treatment of AML” used to quantitate effects of therapy on AML may be any of those criteria defined below, including complete remission (CR) without minimal residual disease (CR_{MRD-}), Complete remission (CR), CR with incomplete hematologic recovery (CRᵢ), Morphologic leukemia-free state (MLFS), Partial remission (PR), stable disease (SD), or progressive disease (PD), as defined by Blood. 2017 Jan. 26; 129(4): 424-447, and summarized as: Complete Remission without minimal residual disease (CR_{MRD-}): If studied pretreatment, CR with negativity for a genetic marker by RT-qPCR, or CR with negativity by MFC; Complete remission (CR): Bone marrow blasts <5%; absence of circulating blasts and blasts with Auer rods; absence of extramedullary disease; absolute neutrophil count (ANC)≥1.0×10⁹/L (1000/μL); platelet count≥100×10⁹/L (100 000/μL); CR with incomplete hematologic recovery (CRᵢ): All CR criteria except for residual neutropenia (<1.0×10⁹/L [1000/μL]) or thrombocytopenia (<100×10⁹/L [100 000/μL]); Morphologic leukemia-free state (MLFS): Bone marrow blasts <5%; absence of blasts with Auer rods; absence of extramedullary disease; no hematologic recovery required; Partial remission (PR): All hematologic criteria of CR; decrease of bone marrow blast percentage to 5% to 25%; and decrease of pretreatment bone marrow blast percentage by at least 50%.

A “combination therapy” is a treatment that includes the administration of two or more therapeutic agents, e.g., a c-KIT inhibitor (such as Compound A or a pharmaceutically acceptable salt thereof, midostaurin, BLU-285, PLX9486, or crenolanib) and a MAPKAP pathway inhibitor (including but not limited to trametinib, cobimetinib, selumetinib,binimetinib, ulixertinib, LY3009120), to a patient. The two or more therapeutic agents may be delivered at the same time, e.g., in separate pharmaceutical compositions or in the same pharmaceutical composition, or they may be delivered at different times. For example, they may be delivered concurrently or during overlapping time periods, and/or one therapeutic agent may be delivered before or after the other therapeutic agent(s). Treatment with a combination of a KIT inhibitor such as Compound A and a MAPKAP pathway inhibitor optionally includes treatment with either single agent, preceded or followed by a period of concurrent treatment with both agents. However, it is contemplated that during some time period, effective amounts of the two or more therapeutic agents are present within the patient.

### Methods of Treatment

In one embodiment, the present disclosure provides methods of treating or preventing a mastocytosis, optionally a c-KIT-mediated mastocytosis, e.g., a systemic mastocytosis (SM), comprising providing to or administering to a subject in need thereof an effective amount of a c-KIT inhibitor in combination with an effective amount of a MAPKAP pathway inhibitor, e.g., trametinib, cobimetinib, selumetinib, binimetinib, ulixertinib, or LY3009120. In one embodiment, the present disclosure provides methods of treating or preventing a mastocytosis, optionally a c-KIT-mediated mastocytosis, e.g., a systemic mastocytosis (SM), comprising providing to or administering to a subject in need thereof an effective amount of Compound A (or a pharmaceutically acceptable salt thereof) or Compound B (or a pharmaceutically acceptable salt thereof), in combination with an effective amount of a a MAPKAP pathway inhibitor, e.g., trametinib, cobimetinib, selumetinib, binimetinib, ulixertinib, or LY3009120. In a related embodiment, the present disclosure provides methods of treating or preventing a mastocytosis tumor, optionally a c-KIT-mediated mastocytosis tumor, e.g., a mast cell leukemia or mast cell sarcoma, comprising providing to or administering to a subject in need thereof an effective amount of a c-KIT inhibitor in combination with an effective amount of a a MAPKAP pathway inhibitor, e.g., trametinib, cobimetinib, selumetinib, binimetinib, ulixertinib, or LY3009120. In a related embodiment, the present disclosure provides methods of treating or preventing a mastocytosis tumor, optionally a c-KIT-mediated mastocytosis tumor, e.g., a mast cell leukemia or mast cell sarcoma, comprising providing to or administering to a subject in need thereof an effective amount of Compound A (or a pharmaceutically acceptable salt thereof) or Compound B (or a pharmaceutically acceptable salt thereof), in combination with an effective amount of a a MAPKAP pathway inhibitor, e.g., trametinib, cobimetinib, selumetinib, binimetinib, ulixertinib, or LY3009120.

In another example, the present disclosure provides a method of treating mastocytosis in a patient in need thereof, comprising administering to the patient: an effective amount of a c-KIT inhibitor; and an effective amount of one or more MAPKAP pathway inhibitors. Such a MAPKAP pathway inhibitor can be selected from the group consisting of a rapidly accelerated fibrosarcoma (RAF) kinase inhibitor, an inhibitor of a mitogen-activated protein kinase (MEK inhibitor), and an extracellular signal regulated kinase inhibitor (ERK inhibitor).

In one embodiment of such a disclosed method, the mastocytosis has a c-KIT mutation. In some embodiments, the c-KIT mutation is an activating mutation.

The mastocytosis may, in another embodiment, comprise mast cells having a primary mutation in exon 17 of a c-KIT gene. In some embodiments, the primary mutation is a c-KIT D816 mutation. In some embodiments, the primary mutation is one of D816V, D816Y, D816F, D816H, F522C, K5091, V560G, V559G, and del419. In some embodiments, the primary mutation is D816V.

The mastocytosis may also comprise mast cells having a secondary c-KIT mutation. In some embodiments, the secondary c-KIT mutation is in one of exon 9, 11, 13 or 17. In some embodiments, the secondary c-KIT mutation is one of Y269C, Y503_F504insAY, V560D, or K642E mutation.

This disclosed method may further comprise determining if the mastocytosis has the c-KIT primary mutation. For example, the method further comprises determining if the mastocytosis has the c-KIT secondary mutation. In some embodiments, determining if the mastocytosis has the c-KIT primary or secondary mutation comprises identifying mutations in DNA extracted from a tumor sample. In yet another embodiment, determining if the mastocytosis has the c-KIT primary or secondary mutation comprises identifying mutations in circulating tumor DNA or from identifying mutations in circulating peripheral blood leukocytes.

The mastocytosis may be systemic mastocytosis. In some embodiments, the systemic mastocytosis is selected from the group consisting of indolent systemic mastocytosis, systemic smoldering mastocytosis, systemic mastocytosis with associated clonal hematological non-mast cell lineage disease, aggressive systemic mastocytosis, mast cell leukemia, and mast cell sarcoma. In some embodiments, the mastocytosis is indolent systemic mastocytosis, optionally systemic mastocytosis with recurrent anaphylaxis or vascular collapse in the absence of skin lesions. In some embodiments, the mastocytosis is systemic smoldering mastocytosis.

In some embodiments, the mastocytosis is systemic mastocytosis with associated clonal hematological non-mast cell lineage disease. In some embodiments, the mastocytosis is aggressive systemic mastocytosis. In some embodiments, the mastocytosis is mast cell leukemia or mast cell sarcoma. In some embodiments, the mastocytosis is cutaneous mastocytosis. In some embodiments, the mastocytosis is selected from the group consisting of: maculopapular cutaneous mastocytosis, mastocytoma, or diffuse cutaneous mastocytosis.

In such a disclosed method, the c-KIT inhibitor can be selected from the group consisting of 1-[4-bromo-5-[1-ethyl-7-(methylamino)-2-oxo-1,2-dihydro-1,6-naphthyridin-3-yl]-2-fluorophenyl]-3-phenylurea or a pharmaceutically acceptable salt thereof, midostaurin or a pharmaceutically acceptable salt thereof, imatinib mesylate, sunitinab malate, midostaurin, regorafenib, crenolanib, PTX9486, or BLU-285 (avapritinib) or a pharmaceutically acceptable salt thereof.

Furthermore, the MEK inhibitor can be selected from the group consisting of trametinib, selumetinib, cobimetinib, and binimetinib. In some embodiments, the MEK inhibitor is binimetinib. In some embodiments, the MEK inhibitor is trametinib. In some embodiments, the ERK inhibitor is selected from the group consisting of ulixertinib, SCH772984, and LY3214996. In some embodiments, the c-KIT inhibitor and the MEK and/or ERK inhibitor are administered substantially concurrently or sequentially.

The method may also comprise administering another cancer-targeted therapeutic agent, cancer-targeted biological, immune checkpoint inhibitor, or chemotherapeutic agent.

Additionally, two weeks or more of administration of an effective amount of a c-KIT inhibitor; and an effective amount of an inhibitor of a mitogen-activated protein kinase (MEK inhibitor), and/or an effective amount of an extracellular signal regulated kinase inhibitor (ERK inhibitor) in accordance with the contemplated method can result in the patient having at least a partial remission.

Also provided is a method of treating a systemic mastocytosis in a patient in need thereof, comprising administering to the patient: an effective amount of 1-[4-bromo-5-[1-ethyl-7-(methylamino)-2-oxo-1,2-dihydro-1,6-naphthyridin-3-yl]-2-fluorophenyl]-3-phenylurea, or a pharmaceutically acceptable salt thereof; and an effective amount of a MAPKAP pathway inhibitor. In such a disclosed method, the MAPKAP pathway inhibitor is selected from the group consisting of rapidly accelerated fibrosarcoma (RAF) kinase inhibitor, an inhibitor of a mitogen-activated protein kinase (MEK inhibitor) and an extracellular signal regulated kinase inhibitor (ERK inhibitor).

The systemic mastocytosis can, in an embodiment, have a c-KIT mutation. For example, the mutation can be a c-KIT D816 mutation. In some embodiments, the mutation is one of D816V, D816Y, D816F, D816H, F522C, K5091, V560G, V559G, and del419. In some embodiments, the mutation is one of: A553D, C433Y, D419Y,D572A, D816F, D816H,D816I, D816V, D816Y, D820G, del419, dup (501-502), E839K, F522C, I817V, InsFF419, InsV815-I816, K5091, N8221, R815K, T417V, V560G, V5591 or Y418Y. In some embodiments, the mutation is D816V.

In addition, the mastocytosis may have a further c-KIT mutation that is one of Y269C, Y503_F504insAY, V560D, or K642E mutation.

In such a disclosed method, the MEK inhibitor can be selected from the group consisting of trametinib, selumetinib, cobimetinib, and binimetinib. In some embodiments, the MEK inhibitor is binimetinib. In some embodiments, the MEK inhibitor is trametinib. The ERK inhibitor can be selected from the group consisting of ulixertinib, SCH772984, and LY3214996.

The present disclosure additionally provides a method of treating mastocytosis in a patient in need thereof, comprising administering to the patient: an effective amount of a c-KIT inhibitor; and an effective amount of a RAF inhibitor.

In such a disclosed method, the RAF inhibitor can be a pan-RAF or B-RAF inhibitor including vemurafenib, dabrafenib, and LY3009120. Furthermore, the c-KIT inhibitor can be 1-[4-bromo-5-[1-ethyl-7-(methylamino)-2-oxo-1,2-dihydro-1,6-naphthyridin-3-yl]-2-fluorophenyl]-3-phenylurea, or a pharmaceutically acceptable salt thereof.

In specific embodiments of methods disclosed herein, including methods of treating a mastocytosis or a mastocytosis tumor, the methods include: induction of cytocidal mast cell killing, induction of apoptosis of mast cells, reduction of the amount of accumulated mast cells in a tissue or organ, reduction of mastocytosis symptoms, inhibition of mast cell mediator release, inhibition of the growth of mast cells, and/or induction of mastocytosis regression, wherein the mast cells harbor one or more activating mutations in c-KIT kinase, such as, e.g., the activating KIT D816V mutation. In certain embodiments, the methods encompass methods for eradicating a mastocytosis, e.g., a mastocytosis tumor, in a subject. In some embodiments, treatment results in a complete response, a partial response, a clinical improvement, or stable disease as defined by IWG-MRT-ECNM criteria (Gotlib et al, Blood 2013; 121: 2393-401).

In another related embodiment, the present disclosure provides methods of treating or preventing an acute myeloid leukemia (AML), optionally a c-KIT-mediated (AML), comprising administering to a subject in need thereof an effective amount of a c-KIT inhibitor in combination with an effective amount of a a MAPKAP pathway inhibitor, e.g., trametinib, cobimetinib, selumetinib, binimetinib, ulixertinib, or LY3009120. In a related embodiment, the present disclosure provides methods of treating or preventing an acute myeloid leukemia (AML), optionally a c-KIT-mediated (AML), comprising administering to a subject in need thereof an effective amount of Compound A (or a pharmaceutically acceptable salt thereof) or Compound B (or a pharmaceutically acceptable salt thereof), in combination with an effective amount of a a MAPKAP pathway inhibitor, e.g., trametinib, cobimetinib, selumetinib, binimetinib, ulixertinib, or LY3009120.

Where the methods described herein refer to treatment with Compound A or a pharmaceutically acceptable salt thereof, or with Compound B or a pharmaceutically acceptable salt thereof, it is meant that only one of Compound A or a pharmaceutically acceptable salt thereof, or Compound B or a pharmaceutically acceptable salt thereof is required. However, it is understood that these methods also encompass administering to a patient both Compound A or a pharmaceutically acceptable salt thereof, and Compound B or a pharmaceutically acceptable salt thereof, in combination with a MAPKAP pathway inhibitor. The methods described herein also encompass administration of Compound A and a MAPKAP pathway inhibitor to a subject whereupon Compound A is metabolized in vivo to Compound B, and the in vivo mixture of Compound A and Compound B effectively treats a subject in combination with the MAPKAP pathway inhibitor.

Particular embodiments of the disclosed methods and compositions are practiced using: a combination of Compound A or a pharmaceutically acceptable salt thereof and trametinib; a combination of Compound A or a pharmaceutically acceptable salt thereof and selumetinib; a combination of Compound A or a pharmaceutically acceptable salt thereof and cobimetinib; or a combination of Compound A or a pharmaceutically acceptable salt thereof and binimetinib.

In one embodiment, Compound A or a pharmaceutically acceptable salt thereof and a MEK inhibitor, e.g., trametinib or binimetinib, are administered to a subject with a c-KIT-mediated mastocytosis. In another embodiment, Compound B or a pharmaceutically acceptable salt thereof and a MEK inhibitor, e.g., trametinib or binimetinib, are administered to a subject with a c-KIT-mediated mastocytosis.

In a related embodiment, Compound A or a pharmaceutically acceptable salt thereof and a MEK inhibitor, e.g., trametinib or binimetinib, are administered to a patient with a mastocytosis tumor, including but not limited to mast cell leukemia or a mast cell sarcoma, wherein mastocytosis tumor growth or tumor progression is caused by a primary activating c-KIT mutation, e.g., the KIT D816V mutation. In another embodiment, Compound B or a pharmaceutically acceptable salt thereof and a MEK inhibitor, e.g., trametinib or binimetinib, are administered to a patient with a mastocytosis tumor, including but not limited to mast cell leukemia or a mast cell sarcoma, wherein mastocytosis tumor growth or tumor progression is caused by a primary activating c-KIT mutation, e.g., the KIT D816V mutation. In certain embodiments, Compound A or a pharmaceutically acceptable salt thereof, or Compound B or a pharmaceutically acceptable salt thereof and a MEK inhibitor, e.g., trametinib or binimetinib, are administered to a patient with AML, optionally wherein the AML is caused by a primary activating c-KIT mutation, e.g., a c-KIT exon 8 activating mutation, or a c-KIT exon 17 mutation, including but not limited to mutations at D816 or at N822 (Journal of Clinical Oncology 2006 24:24, 3904-3911).

Particular embodiments of the disclosed methods and compositions are practiced using: a combination of Compound A or a pharmaceutically acceptable salt thereof and ulixertinib; a combination of Compound A or a pharmaceutically acceptable salt thereof and SCH772984, e; a combination of Compound A or a pharmaceutically acceptable salt thereof and LY3214996,; a combination of Compound A or a pharmaceutically acceptable salt thereof and ravoxertinib, or a combination of Compound A or a pharmaceutically acceptable salt thereof and and VX-11.

In one embodiment, Compound A or a pharmaceutically acceptable salt thereof and an ERK inhibitor, e.g., ulixertinib, are administered to a subject with a c-KIT-mediated mastocytosis. In another embodiment, Compound B or a pharmaceutically acceptable salt thereof and an ERK inhibitor, e.g., ulixertinib, are administered to a subject with a c-KIT-mediated mastocytosis.

In a related embodiment, Compound A or a pharmaceutically acceptable salt thereof and an ERK inhibitor, e.g., ulixertinib, are administered to a patient with a mastocytosis tumor, including but not limited to mast cell leukemia or a mast cell sarcoma, wherein mastocytosis tumor growth or tumor progression is caused by a primary activating c-KIT mutation, e.g., the KIT D816V mutation. In another embodiment, Compound B or a pharmaceutically acceptable salt thereof and an ERK inhibitor, e.g., ulixertinib, are administered to a patient with a mastocytosis tumor, including but not limited to mast cell leukemia or a mast cell sarcoma, wherein mastocytosis tumor growth or tumor progression is caused by a primary activating c-KIT mutation, e.g., the KIT D816V mutation. In certain embodiments, Compound A or a pharmaceutically acceptable salt thereof, or Compound B or a pharmaceutically acceptable salt thereof and an ERK inhibitor, e.g., ulixertinib, are administered to a patient with AML, optionally wherein the AML is caused by a primary activating c-KIT mutation, e.g., a c-KIT exon 8 activating mutation, or a c-KIT exon 17 mutation, including but not limited to mutations at D816 or at N822 (Journal of Clinical Oncology 2006 24:24, 3904-3911).

Particular embodiments of the disclosed methods and compositions are practiced using: a combination of Compound A or a pharmaceutically acceptable salt thereof and LY3009120; a combination of Compound A or a pharmaceutically acceptable salt thereof and dabrafenib; or a combination of Compound A or a pharmaceutically acceptable salt thereof and vemurafenib.

In one embodiment, Compound A or a pharmaceutically acceptable salt thereof and a RAF inhibitor, e.g., LY3009120, dabrafenib, or vemurafenib, are administered to a subject with a c-KIT-mediated mastocytosis. In another embodiment, Compound B or a pharmaceutically acceptable salt thereof and a RAF inhibitor, e.g., LY3009120, dabrafenib, or vemurafenib, are administered to a subject with a c-KIT-mediated mastocytosis. In a related embodiment, Compound A or a pharmaceutically acceptable salt thereof and a RAF inhibitor, e.g., LY3009120, dabrafenib, or vemurafenib, are administered to a patient with a mastocytosis tumor, including but not limited to mast cell leukemia or a mast cell sarcoma, wherein mastocytosis tumor growth or tumor progression is caused by a primary activating c-KIT mutation, e.g., the KIT D816V mutation. In another embodiment, Compound B or a pharmaceutically acceptable salt thereof and a RAF inhibitor, e.g., LY3009120, dabrafenib, or vemurafenib, are administered to a patient with a mastocytosis tumor, including but not limited to mast cell leukemia or a mast cell sarcoma, wherein mastocytosis tumor growth or tumor progression is caused by a primary activating c-KIT mutation, e.g., the KIT D816V mutation. In certain embodiments, Compound A or a pharmaceutically acceptable salt thereof, or Compound B or a pharmaceutically acceptable salt thereof and a RAF inhibitor, e.g., LY3009120, dabrafenib, or vemurafenib, are administered to a patient with AML, optionally wherein the AML is caused by a primary activating c-KIT mutation, e.g., a c-KIT exon 8 activating mutation, or a c-KIT exon 17 mutation, including but not limited to mutations at D816 or at N822 (Journal of Clinical Oncology 2006 24:24, 3904-3911).

Illustrative c-KIT inhibitors that may be used according to the disclosed methods and compositions include, but are not limited to, Compound A or a pharmaceutically acceptable salt there, Compound B or a pharmaceutically acceptable salt thereof, midostaurin, BLU-285, PLX9486, and crenolanib. Illustrative MEK inhibitors that may be used according to the disclosed methods and compositions include, but are not limited to, trametinib, selumetinib, cobimetinib, and binimetinib. Illustrative ERK inhibitors that may be used according to the disclosed methods and compositions include, but are not limited to, ulixertinib, SCH772984, LY3214996, ravoxertinib, and VX-11e. Illustrative RAF inhibitors that may be used according to the disclosed methods and compositions include, but are not limited to, LY3009120, dabrafenib, and vemurafenib.

Treatment with Compound A or a pharmaceutically acceptable salt thereof, or Compound B or a pharmaceutically acceptable salt thereof, in combination with a MAPKAP pathway inhibitor, e.g., trametinib, binimetinib, ulixertinib, or LY3009120, encompasses administering Compound A or a pharmaceutically acceptable salt thereof, or Compound B or a pharmaceutically acceptable salt thereof, before, after, simultaneous with, or during an overlapping time period with administering the MAPKAP pathway inhibitor. It is understood that an effective amount of any of Compound A or a pharmaceutically acceptable salt thereof, Compound B or a pharmaceutically acceptable salt thereof, another c-KIT inhibitor, or a MAPKAP pathway inhibitor, e.g., trametinib, binimetinib, ulixertinib, or LY3009120, may be different when used in the combinations disclosed herein as compared to when any of these agents is used by itself for the same purpose, e.g., to treat or prevent mastocytosis or a mast cell tumor, e.g., a mast cell leukemia or AML. In particular embodiments, an effective amount of Compound A or a pharmaceutically acceptable salt thereof, or of Compound B or a pharmaceutically acceptable salt thereof, is a lower amount when administered as a combination therapy with a MAPKAP pathway inhibitor, e.g., trametinib, binimetinib, ulixertinib, or LY3009120, as compared to when it is administered as a monotherapy, e.g., to treat or prevent a mastocytosis or a mast cell tumor. In particular embodiments, an effective amount of a MAPKAP pathway inhibitor, e.g., trametinib, binimetinib, ulixertinib, or LY3009120, is a lower amount when administered in a combination therapy with Compound A or a pharmaceutically acceptable salt thereof, or when administered in a combination therapy with Compound B or a pharmaceutically acceptable salt thereof, e.g., to treat or prevent a mastocytosis or a mast cell tumor.

Any of the methods disclosed herein may further include determining that the mastocytosis cells, mast cell tumor, or AML being treated has one or more c-KIT gene mutations. Such a determination may be made by routine methods for determining the presence of a gene mutation in a biological sample, e.g., a bone marrow sample, a tissue sample, a peripheral blood sample, or a plasma sample, obtained from the subject. In addition, such a determination may be made by reviewing the results of tests performed to determine the presence of one or more c-KIT gene mutations in the biological sample obtained from the patient. In certain embodiments of any of the methods disclosed herein, the methods are performed on subjects wherein the mastocytosis, mast cell tumor, or AML has been identified as having one or more c-KIT gene mutations. The c-KIT gene mutations include but not limited to any of those specifically described herein. In certain embodiments of any of the methods disclosed herein, the methods are not performed on subjects wherein the mastocytosis, mast cell tumor, or AML has been identified as not having one or more c-KIT gene mutations.

In various aspects of any of the methods disclosed herein, treatment with either Compound A or a pharmaceutically acceptable salt thereof, or Compound B or a pharmaceutically acceptable salt thereof, in combination with a MAPKAP pathway inhibitor, e.g., trametinib, binimetinib, ulixertinib, or LY3009120, induces cytocidal mast cell killing, induces apoptosis of mast cells, reduces the amount of accumulated mast cells in a tissue or organ, reduces mastocytosis symptoms, inhibits mast cell mediator release, inhibits the growth of mast cells, and/or induces mastocytosis regression wherein mast cells harbor activating mutations in c-KIT kinase including the activating KIT D816V mutation. Methods for measuring or determining amounts of apoptosis of mast cells, mast cell killing, inhibition of mast cell growth and proliferation, inhibition of mast cell mediator release, peripheral blood mutant KIT allele burden, eradication of mastocytosis and mastocytosis tumors, complete response, partial response, clinical improvement, or stable disease are known in the art and include any methods described herein.

In particular embodiments, treatment with a combination of: a c-KIT inhibitor, e.g., Compound A or a pharmaceutically acceptable salt thereof, or Compound B or a pharmaceutically acceptable salt thereof; and a MAPKAP pathway inhibitor, e.g., trametinib, binimetinib, ulixertinib, or LY3009120, results in an increased amount of apoptosis of mastocytosis cells or mast cells, as compared to the amount of apoptosis of mastocytosis cells or mast cells of the same type either untreated or treated with only a MAPKAP pathway inhibitor, or with only a c-KIT inhibitor, e.g., Compound A or Compound B. For example, apoptosis may be increased by at least 10%, at least 20%, at least 30%, at least 40%, at least 50%, at least 60%, at least 70%, at least 80%, at least 90%, at least two-fold, at least three-fold, at least four-fold, at least five-fold, at least 10-fold, or at least 20-fold. In certain embodiments, amounts of apoptosis are determined by measuring caspase activity of KIT mutant mast cells or mast cell lines including the HMC1.2 mast cell line harboring the KIT D816V mutation.

In particular embodiments, treatment with a combination of: a c-KIT unhibitor, e.g., Compound A or a pharmaceutically acceptable salt thereof, or Compound B or a pharmaceutically acceptable salt thereof; and a MAPKAP pathway inhibitor, e.g., trametinib, binimetinib, ulixertinib, or LY3009120, results in a decreased accumulation of mast cells in skin or an internal organ, e.g., liver, spleen, bone marrow, and/or small intestine, as compared to the amount of accumulation of mast cells in the same organ either untreated or treated with only a MAPKAP pathway inhibitor, e.g., trametinib, binimetinib, ulixertinib, or LY3009120, or with only a c-KIT inhibitor, e.g., midostaurin, BLU-285, Compound A or Compound B. For example, the amount or number of mast cells accumulated within the organ may be decreased by at least 10%, at least 20%, at least 30%, at least 40%, at least 50%, at least 60%, at least 70%, at least 80%, or at least 90%.

In particular embodiments, treatment with a combination of: a c-KIT inhibitor, e.g., Compound A or a pharmaceutically acceptable salt thereof, or Compound B or a pharmaceutically acceptable salt thereof; and a MAPKAP pathway inhibitor, e.g., trametinib, binimetinib, ulixertinib, or LY3009120, results in a complete response as defined by as defined by IWG-MRT-ECNM criteria (Gotlib et al, Blood 2013; 121 :2393-401).

In particular embodiments, treatment with a combination of: a c-KIT inhibitor, e.g., Compound A or a pharmaceutically acceptable salt thereof, or Compound B or a pharmaceutically acceptable salt thereof; and a MAPKAP pathway inhibitor, e.g., trametinib, binimetinib, ulixertinib, or LY3009120, results in a partial response as defined by as defined by IWG-MRT-ECNM criteria (Gotlib et al, Blood 2013; 121: 2393-401).

In particular embodiments, treatment with a combination of: a c-KIT inhibitor,

e.g., Compound A or a pharmaceutically acceptable salt thereof, or Compound B or a pharmaceutically acceptable salt thereof; and a MAPKAP pathway inhibitor, e.g., trametinib, binimetinib, ulixertinib, or LY3009120, results in clinical improvement as defined by as defined by IWG-MRT-ECNM criteria (Gotlib et al, Blood 2013; 121: 2393-401).

In particular embodiments, treatment with a combination of: a c-KIT inhibitor, e.g., Compound A or a pharmaceutically acceptable salt thereof, or Compound B or a pharmaceutically acceptable salt thereof; and a MAPKAP pathway inhibitor, e.g., trametinib, binimetinib, ulixertinib, or LY3009120, results in stable disease as defined by as defined by IWG-MRT-ECNM criteria (Gotlib et al, Blood 2013; 121:2393-401).

In particular embodiments, treatment with a combination of: a c-KIT inhibitor, e.g., Compound A or a pharmaceutically acceptable salt thereof, or Compound B or a pharmaceutically acceptable salt thereof; and a MAPKAP pathway inhibitor, e.g., trametinib, binimetinib, ulixertinib, or LY3009120, induces apoptosis or inhibits the growth of resistant mastocytosis cells containing a secondary KIT mutation in addition to the KIT D816V primary mutation wherein the secondary KIT mutation includes but is not limited to Y269C, Y503_F504insAY, V560D, or K642E point mutation, an in-frame deletion or insertion, or a missense mutation in the c-KIT gene (Lasho et al, Br J Haematol 173, 153-156).

In particular embodiments, treatment with a combination of: a c-KIT inhibitor, e.g., Compound A or a pharmaceutically acceptable salt thereof, or Compound B or a pharmaceutically acceptable salt thereof; and a MAPKAP pathway inhibitor, e.g., trametinib, binimetinib, ulixertinib, or LY3009120, induces apoptosis or inhibits the growth of resistant mastocytosis cells containing mutations, e.g., resistance mutations, in other genes, including any of those disclosed herein. In certain embodiments, such other gene mutations may include NRas gain of function mutations (Haematologica 2011;96(03):459-463.doi:10.3324/haematol.2010.031690) or TET2 loss of function mutations (Leukemia 2009;23:900-04; Blood, 6 Dec. 2012; 120 (24): 4846-49). The presence of other epigenetic or transcriptional regulator mutations have been detected in SM, including DNMT3A, ASXL1 and CBL mutations in 12%, 12% and 4% of patients, respectively (PloS one. 2012;7:e43090). Additionally, some mastocytosis patients also present with mutations in the spliceosome machinery. Spliceosomes ensure the correct linear order of exons spliced in mRNAs. Hanssens et al. reported 23.6% SRSF2, 5.6% SF3B1 and 2.7% U2AF1 incidence of mutations in a group of 72 mastocytosis patients (Haematologica 2014; 99:830-35). Such mastocytoses having complex genomic drivers may benefit from a treatment disclosed herein with a combination of a c-KIT inhibitor and a MEK inhibitor as compared to no treatment or treatment with only a MEK inhibitor, e.g., trametinib, or with only a c-KIT inhibitor, e.g., Compound A or Compound B. For example, apoptosis may be increased by at least 10%, at least 20%, at least 30%, at least 40%, at least 50%, at least 60%, at least 70%, at least 80%, at least 90%, at least two-fold, at least three-fold, at least four-fold, at least five-fold, at least 10-fold, or at least 20-fold. For example, the amount of growth or number of resistant mastocytosis cells may be inhibited by at least 10%, at least 20%, at least 30%, at least 40%, at least 50%, at least 60%, at least 70%, at least 80%, or at least 90%.

In particular embodiments, resistant mastocytosis cells are resistant to apoptosis-mediated cell death or cytocidal activity by treatment with a c-KIT inhibitor, e.g., Compound A, Compound B, midostaurin, BLU-285, PLX9486, or crenolanib, and/or are resistant to apoptosis-mediated cell death or cytocidal activity by treatment with a MAPKAP pathway inhibitor, e.g., trametinib, binimetinib, ulixertinib, or LY3009120, when used as single agent therapies.

In particular embodiments, treatment with a combination of: a c-KIT inhibitor, e.g., either Compound A or a pharmaceutically acceptable salt thereof, or Compound B or a pharmaceutically acceptable salt thereof; in combination with a MAPKAP pathway inhibitor, e.g., trametinib, binimetinib, ulixertinib, or LY3009120, results in eradication of a mastocytosis, e.g., a matocytosis tumor. In particular embodiments, eradication of a mastocytosis means there is no longer any detectable mastocytosis in the patient. In particular embodiments, there is no detectable mastocytosis in the patient for at least twelve weeks, at least twenty-four weeks, at least one year, at least two years, or at least 5 years after initiation of treatment of the mastocytosis by a combination therapy disclosed herein. Eradication of mastocytosis may be determined by criteria for complete response as defined by IWG-MRT-ECNM criteria (Gotlib et al, Blood 2013; 121: 2393-401).

The present disclosure describes combination therapies that involve the administration of a c-KIT inhibitor, e.g., either Compound A or a pharmaceutically acceptable salt thereof, or Compound B or a pharmaceutically acceptable salt thereof, and a MAPKAP pathway inhibitor, e.g., trametinib, binimetinib, ulixertinib, or LY3009120. The combination therapies described herein can be used by themselves, or in combination with one or more additional therapeutic agents. For example, either Compound A or a pharmaceutically acceptable salt thereof or Compound B or a pharmaceutically acceptable salt thereof, and a MAPKAP pathway inhibitor, can be administered together with a cancer targeted therapeutic agent, a cancer-targeted biologic, an immune checkpoint inhibitor, or a chemotherapeutic agent. In another embodiment, Compound A or Compound B and a MAPKAP pathway inhibitor, e.g., trametinib, binimetinib, ulixertinib, or LY3009120are administered without any other therapeutic agent. The therapeutic agents can be administered together with or sequentially with another therapeutic agent described herein in a combination therapy.

Combination therapy can be achieved by administering two or more therapeutic agents, each of which is formulated and administered separately, or by administering two or more therapeutic agents in a single formulation. Other combinations are also encompassed by combination therapy. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination using different sequencing of the component agents. For example, if agent X and agent Y are used in a combination, one could administer them sequentially in any combination one or more times, e.g., in the order X-Y-X, X-X-Y, Y-X-Y, Y-Y-X, X-X-Y-Y, etc. Furthermore, administration of two or more agents of the combination may precede or follow administration dosing intervals during which at least one of the combination agents is omitted from the treatment.

Additional therapeutic agents that may be administered according to the present disclosure, e.g., to treat a mastocytosis or mastocytosis tumor, include, but are not limited to, agents selected from the group consisting of: inhibitors of STAT5, including but not limited to ruxolitinib, tofacitinib, or fedratinib; inhibitors of BTK including but not limited to ibrutinib, PCI 29732, acalabrutinib, or AVL-292; inhibitors of PI3 kinase including but not limited to idelalisib, dactolisib, pictilisib, LY294002, buparlisib, pilaralisib, duvelisib, PF-04691502, voxtalisib, omipalisib, gedatolisib, apitolisib, or wortmannin; inhibitors of AKT kinase including but not limited to MK-2206, perifosine, GSK690693, GSK2141795, ipatasertib, AZD5363, afuresertib, or AT7867; inhibitors of DNA methylation including but not limited to 5-azacytidine or 5-aza-2′-deoxycytidine; proteosomal inhibitors including but not limited to bortezomib, carfilzomib, MLN9708, ONX 0912;; interferon-alpha (IFN-□); cladribine; and lysosomotropic agents including but not limited to chloroquine, hydroxychloroquine, or quinacrine.

In certain embodiments, the additional therapeutic agent is selected from 5-azacytidine, 5-aza-2′-deoxycytidine, and cladribine.

In particular embodiments, a subject in need thereof is treated with a combination of Compound A or a pharmaceutically acceptable salt thereof, one or two of a MAPKAP pathway inhibitor, e.g., trametinib, binimetinib, ulixertinib, or LY3009120, and 5-azacytidine. In certain embodiments, the MAPKAP pathway inhibitor is trametinib. In certain embodiments, the MAPKAP pathway inhibitor is binimetinib. In certain embodiments, the MAPKAP pathway inhibitor is ulixertinib. In certain embodiments, the MAPKAP pathway inhibitor is LY3009120, dabrafenib or vemurafenib.

In particular embodiments, a subject in need thereof is treated with a combination of Compound A or a pharmaceutically acceptable salt thereof, a MAPKAP pathway inhibitor, e.g., trametinib, binimetinib, ulixertinib, or LY3009120, and 5-aza-2′-deoxycytidine. In certain embodiments, the MAPKAP pathway inhibitor is trametinib. In certain embodiments, the MAPKAP pathway inhibitor is binimetinib. In certain embodiments, the MAPKAP pathway inhibitor is ulixertinib. In certain embodiments, the MAPKAP pathway inhibitor is LY3009120, dabrafenib or vemurafenib.

In particular embodiments, a subject in need thereof is treated with a combination of Compound A or a pharmaceutically acceptable salt thereof, MAPKAP pathway inhibitor, e.g., trametinib, binimetinib, ulixertinib, or LY3009120, and cladribine. In certain embodiments, the MAPKAP pathway inhibitor is trametinib. In certain embodiments, the MAPKAP pathway inhibitor is binimetinib. In certain embodiments, the MAPKAP pathway inhibitor is ulixertinib. In certain embodiments, the MAPKAP pathway inhibitor is LY3009120, dabrafenib or vemurafenib. Additional therapeutic agents that may be administered according to the present disclosure include, but are not limited to, arsenic trioxide, cyclophosphamide, cytarabine, daunorubicin, doxorubicin, enasidenib, idarubicin, quizartinib, mitoxantrone, thioguanine, or vincristine. In particular embodiments, a subject having AML is treated with a combination of Compound A or a pharmaceutically acceptable salt thereof,, and arsenic trioxide, cyclophosphamide, cytarabine, daunorubicin, doxorubicin, enasidenib, idarubicin, quizartinib, mitoxantrone, thioguanine, or vincristine.

### Pharmaceutical Compositions

Aspects of the present disclosure are directed to methods of treatment involving the administration of a combination of compounds disclosed herein, or one or more pharmaceutical compositions comprising such compounds and a pharmaceutically acceptable diluent, excipient or carrier. In particular embodiments, the methods disclosed herein involve administering a first pharmaceutical composition comprising a c-KIT inhibitor, e.g., Compound A or a pharmaceutically acceptable salt thereof and a pharmaceutically acceptable diluent, excipient or carrier, and a second pharmaceutical composition comprising a MAPKAP pathway inhibitor, e.g., trametinib, binimetinib, ulixertinib, or LY3009120, and a pharmaceutically acceptable diluent, excipient or carrier. In particular embodiments, the methods disclosed herein involve administering a first pharmaceutical composition comprising Compound B or a pharmaceutically acceptable salt thereof and a pharmaceutically acceptable diluent, excipient or carrier, and a second pharmaceutical composition comprising a MAPKAP pathway inhibitor, e.g., trametinib, binimetinib, ulixertinib, or LY3009120, and a pharmaceutically acceptable diluent, excipient or carrier. In particular embodiments, the methods disclosed herein involve administering a pharmaceutical composition comprising a ciKIT inhibitor, e.g., Compound A or a pharmaceutically acceptable salt thereof, a MAPKAP pathway inhibitor, e.g., trametinib, binimetinib, ulixertinib, or LY3009120, and a pharmaceutically acceptable diluent, excipient or carrier. In particular embodiments, the methods disclosed herein involve administering a pharmaceutical composition comprising Compound B or a pharmaceutically acceptable salt thereof, a MAPKAP pathway inhibitor, e.g., trametinib, binimetinib, ulixertinib, or LY3009120, and a pharmaceutically acceptable diluent, excipient or carrier.

In using the pharmaceutical compositions of the compounds described herein, pharmaceutically acceptable carriers can be either solid or liquid. Solid forms include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa, which is hereby incorporated by reference in its entirety.

Liquid form preparations include solutions, suspensions and emulsions. For example, water or water-propylene glycol solutions for parenteral injection or addition of sweeteners and opacifiers for oral solutions, suspensions and emulsions. Liquid form preparations may also include solutions for intranasal administration.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Aerosol preparations suitable for inhalation may also be used. These preparations may include solutions and solids in powder form, which may be in combination with a pharmaceutically acceptable carrier, such as an inert compressed gas, e.g., nitrogen.

Also contemplated for use are solid form preparations that are intended to be converted, shortly before use, to liquid form preparations for either oral or parenteral administration. Such liquid forms include solutions, suspensions and emulsions.

### Dosage

In some embodiments where Compound A or Compound B (or pharmaceutically acceptable salts thereof) is used in combination with a MAPKAP pathway inhibitor, e.g., trametinib, binimetinib, ulixertinib, or LY3009120 for a treatment protocol, the two therapeutics may be administered together or in a “dual-regimen” wherein the two therapeutics are dosed and administered separately. When the Compound A or B (or pharmaceutically acceptable salts thereof) and the MAPKAP pathway inhibitor are dosed separately, the typical dosage of Compound A or Compound B (or pharmaceutically acceptable salts thereof) administered to the subject in need of the treatment is typically from about 5 mg per day to about 5000 mg per day and, in other embodiments, from about 50 mg per day to about 1000 mg per day. Other dosages may be from about 10 mmol up to about 250 mmol per day, from about 20 mmol to about 70 mmol per day or even from about 30 mmol to about 60 mmol per day. Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. A typical recommended daily dosage regimen for oral administration can range from about 1 mg/day to about 500 mg/day or 1 mg/day to 200 mg/day, in a single dose, or in two to four divided doses. In one embodiment, the typical daily oral dose regimen is 150 mg.

In certain embodiments, the dosage of MAPKAP pathway inhibitors is consistent with previously disclosed dosages and/or dosages approved for use by the Food and Drug Administration. In other embodiments, the dosage of the MAPKAP pathway inhibitor is less than previously approved dosages, e.g., about 20%, about 50% or about 80% of an approved dosage. In certain embodiments, the dosage of trametinib is about 0.5 mg to 20 mg orally daily, e.g., about 1 mg daily or about 2 mg daily. In certain embodiments, the dosage of cobimetinib is about 10 mg to 200 mg daily, e.g., about 30 mg or about 60 mg daily. In certain embodiments, the dosage of binimetinib is about 10 mg to about 200 mg twice daily, e.g., about 25 mg or about 45 mg twice daily. In certain embodiments, the dosage of selumetinib is about 10 mg to about 200 mg daily, or about 30 mg or about 75 mg twice daily.

The amount and frequency of administration of the compounds described herein and/or the pharmaceutically acceptable salts thereof, and other therapeutic agents, will be regulated according to the judgment of the attending clinician considering such factors as age, condition and size of the patient as well as severity of the symptoms being treated.

Compounds of the present disclosure (e.g., Compound A or Compound B (and pharmaceutically acceptable salts thereof), MAPKAP pathway inhibitors, and other therapeutic agents) may be administered by any suitable route. The compounds can be administrated orally (e.g., dietary) in capsules, suspensions, tablets, pills, dragees, liquids, gels, syrups, slurries, and the like. Methods for encapsulating compositions (such as in a coating of hard gelatin or cyclodextran) are known in the art (Baker, et al., “Controlled Release of Biological Active Agents”, John Wiley and Sons, 1986, which is hereby incorporated by reference in its entirety). The compounds can be administered to the subject in conjunction with an acceptable pharmaceutical carrier as part of a pharmaceutical composition. The formulation of the pharmaceutical composition will vary according to the route of administration selected. Suitable pharmaceutical carriers may contain inert ingredients which do not interact with the compound. The carriers are biocompatible, i.e., non-toxic, non-inflammatory, non-immunogenic and devoid of other undesired reactions at the administration site.

Illustrative pharmaceutical compositions are tablets and gelatin capsules comprising a Compound described herein, e.g., Compound A or a pharmacetucially acceptable salt thereof, and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

If formulated as a fixed dose, such combination products employ the compounds described herein within the dosage range described herein, or as known to those skilled in the art.

Since the compounds described herein (e.g., Compounds A and B and MAPKAP pathway inhibitors) are intended for use in pharmaceutical compositions a skilled artisan will understand that they can be provided in substantially pure forms for example, at least 60% pure, at least 75% pure, at least 85% pure, and at least 98% pure (w/w). The pharmaceutical preparation may be in a unit dosage form. In such form, the preparation is subdivided into suitably sized unit doses containing appropriate quantities of compounds A or B, e.g., an effective amount to achieve the desired purpose as described herein.

### EXAMPLES

It has been found that treatment with a combination of either Compound A or a pharmaceutically acceptable salt thereof in combination with a MAPKAP pathway inhibitor unexpectedly and synergistically induce apoptosis of mutant c-KIT-driven mast cells causative of systemic mastocytosis. In addition, this combination therapy inhibits proliferation of mastocytosis cells. Furthermore, the combination therapy disclosed herein appeared to have a cytotoxic effect on mastocytosis, as opposed to merely a cytostatic effect as determined by combination treatment-induced in caspase activation. Characterization of this unexpected finding was undertaken in biochemical assays and cellular assays, including those described herein.

The disclosure is thus further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1. Treatment of Mutant KIT Mast Cell Lines with Compound A or Compound B Inhibits Cell Proliferation and KIT Phosphorylation of Mastocytosis Cell Lines

A study was performed that demonstrates that treatment with Compound A or Compound B inhibited cell proliferation of mutant KIT HMC1.1 KIT V560G and HMC1.2 KIT V560G/D816V mast cell lines. Assays were conducted in 96 well plates with 10,000 cells seeded per well. The cells were treated with vehicle control, Compound A, or Compound B thereof at varying concentrations, allowed to grow for 72 hours, and then cell proliferation was assessed.

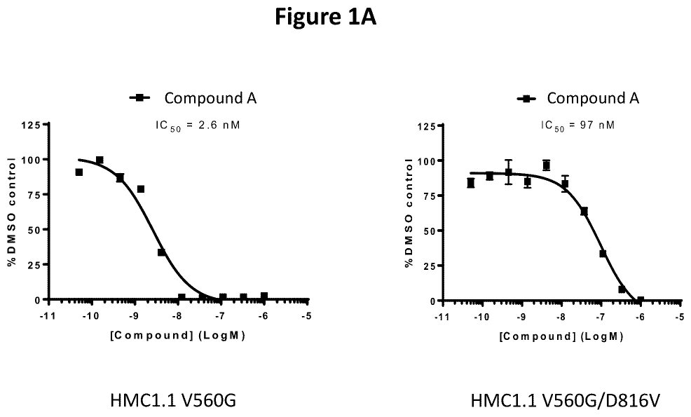

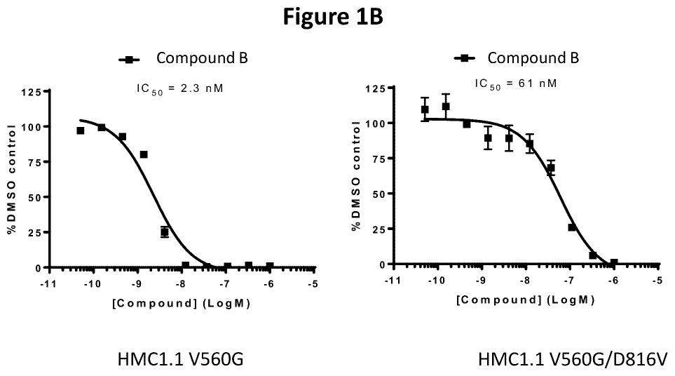

FIG. 1A is a graphical representation showing the relative percentage of cell proliferation determined for various concentrations of Compound A. Treatment with Compound A inhibited cell proliferation growth in HMC1.1 V560G and HMC1.2 V560G/D816V mast cell lines with IC50 values of 2.6 nM and 97 nM, respectively.

FIG. 1B is a graphical representation showing the relative percentage of cell proliferation determined for various concentrations of Compound B. Treatment with Compound B inhibited cell proliferation growth in HMC1.1 V560G and HMC1.2 V560G/D816V cell lines with IC50 values of 2.3 nM and 61 nM, respectively.

### Example 2. Combination Treatment with Compound A and Trametinib Induces Apoptosis in Mastocytosis Cell Lines

A study was performed to demonstrate that combination treatment with Compound A and the MEK inhibitor trametinib induced apoptosis in the HMC1.2 KIT V560G/D816V mastocytosis cell line. Assays were conducted in 96 well plates with 10,000 cells seeded per well. Cells were treated with vehicle control, Compound A, trametinib, or combinations thereof at varying concentrations, and the cells were allowed to grow for 24 hours. Apoptosis was assessed by measuring caspase 3/7 activity.

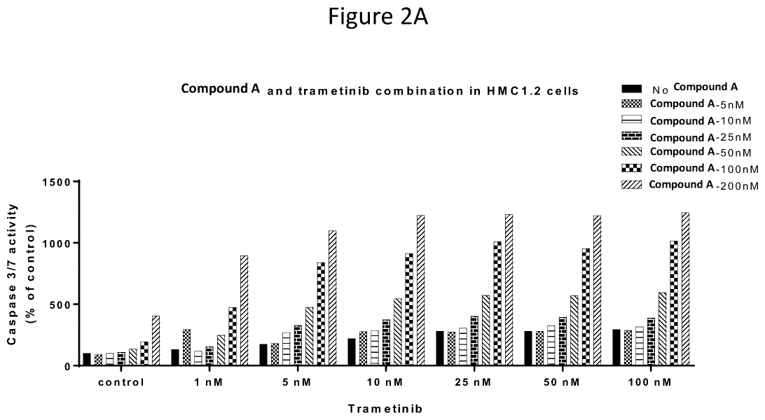

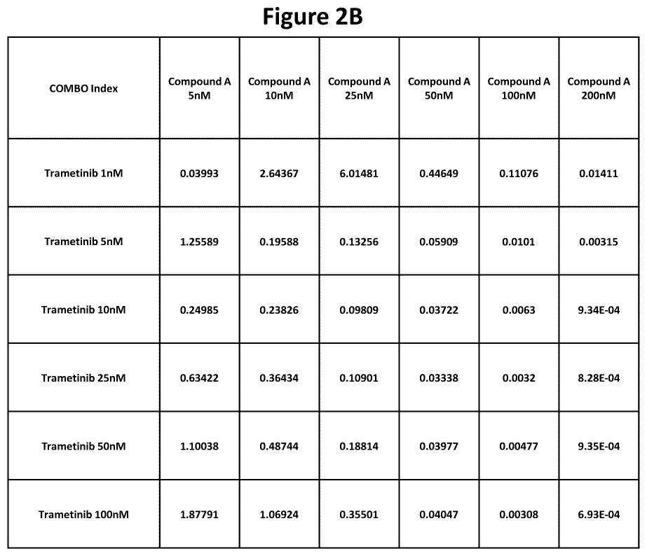

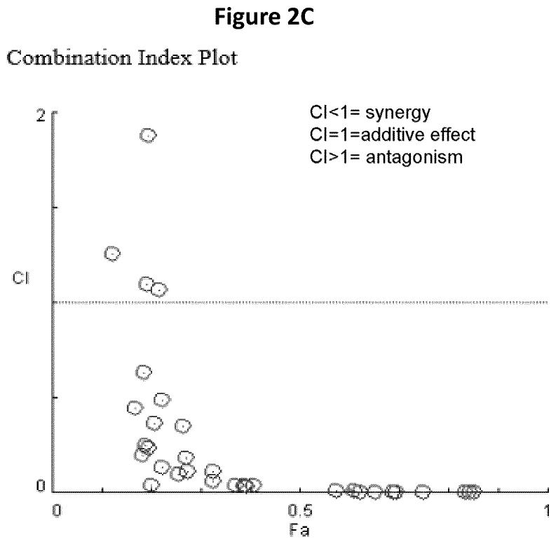

FIG. 2A is graphical representation showing the relative percentage of cell apoptosis determined for various treatments. FIG. 2B is a matrix for synergy chart based on the combination index (CI) method described by Chou and Talalay (1984) and the computer software of Chou and Martin (2005). CI<1 indicates synergism, CI=1 indicates additive effect, and CI>1 indicates antagonism. Combination treatments with Compound A and the MEK inhibitor trametinib unexpectedly showed strong synergy for inducing apoptosis of HMC1.2 KIT V560G/D816V cells. FIG. 2C is a Combination Index Plot of the CI, demonstrating strong synergy for combination of Compound A with trametinib for inducing apoptosis of HMC1.2 KIT V560G/D816V mast cells.

### Example 3. Combination Treatment with Compound B and Trametinib Induces Apoptosis in Mastocytosis Cell Lines

A study was also performed to demonstrate that combination treatment with Compound B and trametinib induced apoptosis in the HMC1.2 KIT V560G/D816V mastocytosis cell line. Assays were conducted as explained in example 2. Apoptosis was assessed by measuring caspase 3/7 activity.

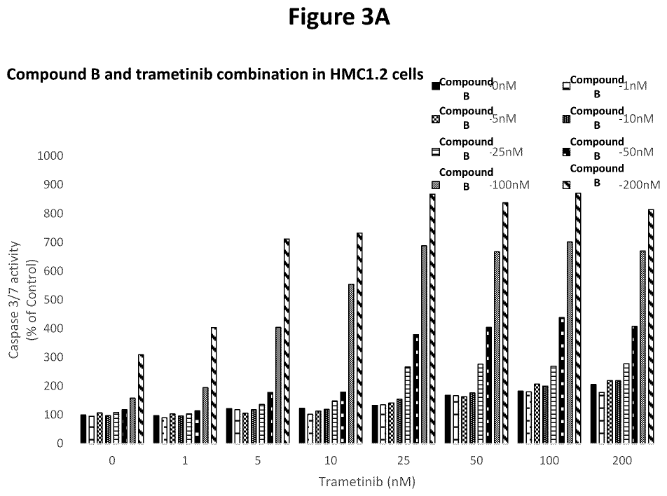

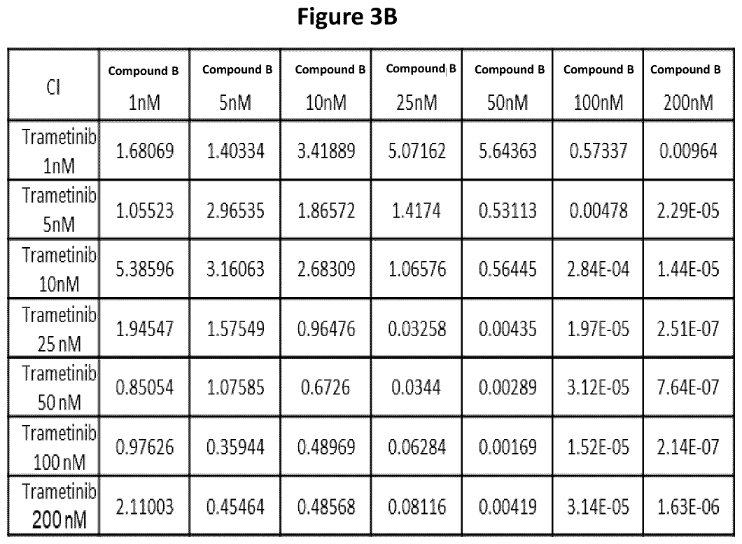

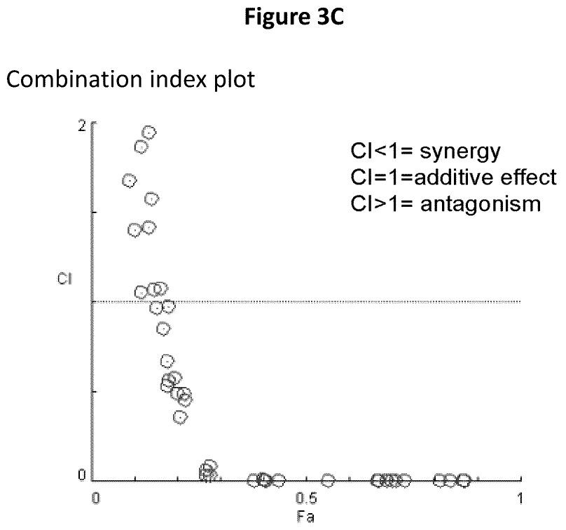

FIG. 3A is graphical representation showing the relative percentage of cell apoptosis determined for various treatments. FIG. 3B is a matrix for synergy chart based on the combination index (CI) method described by Chou and Talalay (1984) and the computer software of Chou and Martin (2005). CI<1 indicates synergism, CI=1 indicates additive effect, and CI>1 indicates antagonism. Combination treatments with Compound B and the MEK inhibitor trametinib unexpectedly showed strong synergy for inducing apoptosis of HMC1.2 KIT V560G/D816V cells. FIG. 3C is a Combination Index Plot of the CI, demonstrating strong synergy for combination of Compound B with trametinib for inducing apoptosis of HMC1.2 KIT V560G/D816V mast cells.

### Example 4. Combination Treatment with Compound A and Binimetinib Induces Apoptosis in Mastocytosis Cell Lines

Combination treatment with Compound A and binimetinib also induced apoptosis in the HMC1.2 KIT V560G/D816V mastocytosis cell line. Assays were conducted as explained in example 2. Apoptosis was assessed by measuring caspase 3/7 activity.

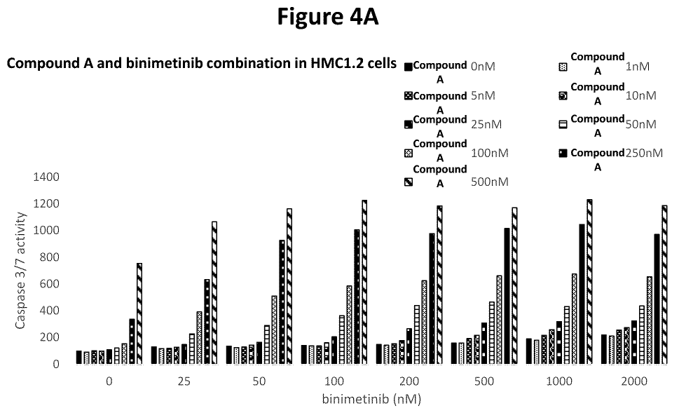

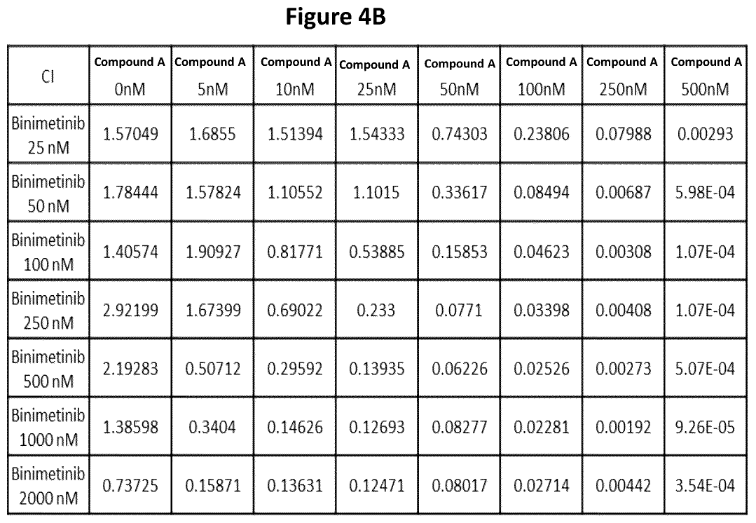

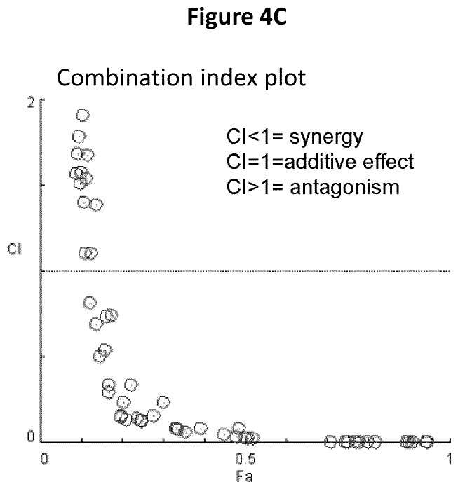

FIG. 4A is graphical representation showing the relative percentage of cell apoptosis determined for various treatments. FIG. 4B is a matrix for synergy chart based on the combination index (CI) method described by Chou and Talalay (1984) and the computer software of Chou and Martin (2005). Combination treatments with Compound A and the MEK inhibitor binimetinib unexpectedly showed strong synergy for inducing apoptosis of HMC1.2 KIT V560G/D816V cells. FIG. 4C is a Combination Index Plot of the CI, demonstrating strong synergy for combination of Compound A with binimetinib for inducing apoptosis of HMC1.2 KIT V560G/D816V mast cells.

### Example 5. Combination Treatment with Compound B and Binimetinib Induces Apoptosis in Mastocytosis Cell Lines

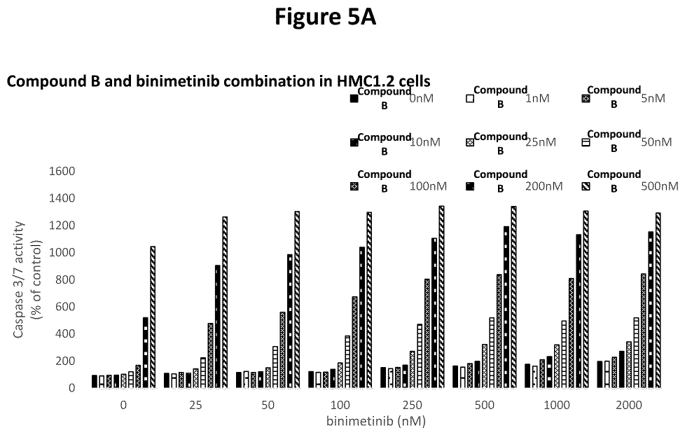

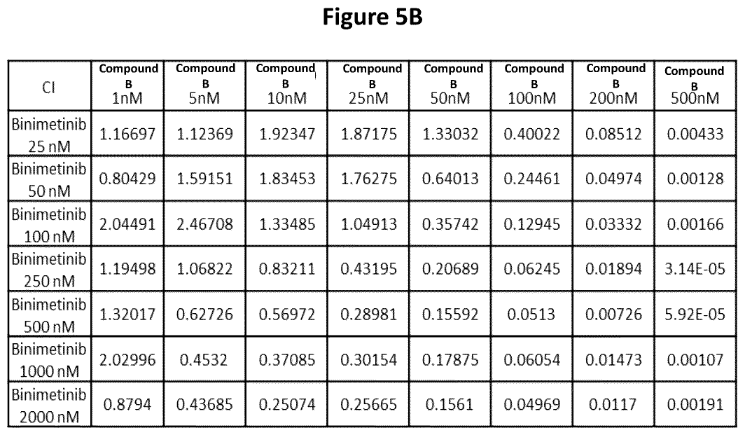

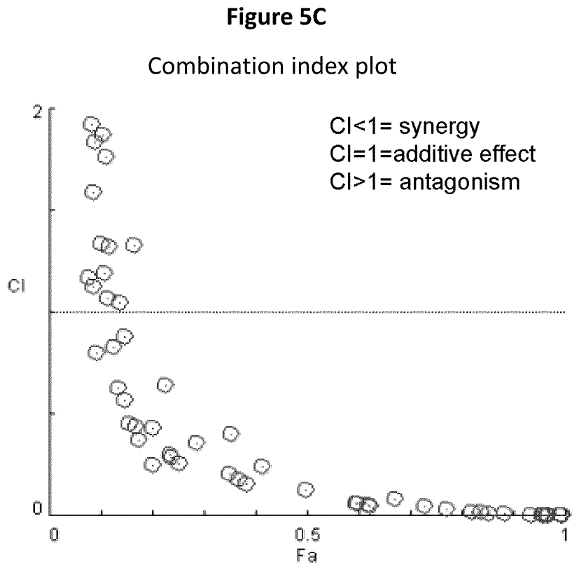

FIG. 5A is graphical representation showing the relative percentage of cell apoptosis determined for various treatments of Compound B and binimetinib. FIG. 5B is a matrix for synergy chart based on the combination index (CI) method described by Chou and Talalay (1984) and the computer software of Chou and Martin (2005). CI<1 indicates synergism, CI=1 indicates additive effect, and CI>1 indicates antagonism. Combination treatments with Compound B and the MEK inhibitor binimetinib unexpectedly showed strong synergy for inducing apoptosis of HMC1.2 KIT V560G/D816V cells. FIG. 5C is a Combination Index Plot of the CI, demonstrating strong synergy for combination of Compound B with binimetinib for inducing apoptosis of HMC1.2 KIT V560G/D816V mast cells.

### Example 6. Combination Treatment with Compound A and Cobimetinib Induces Apoptosis in Mastocytosis Cell Lines

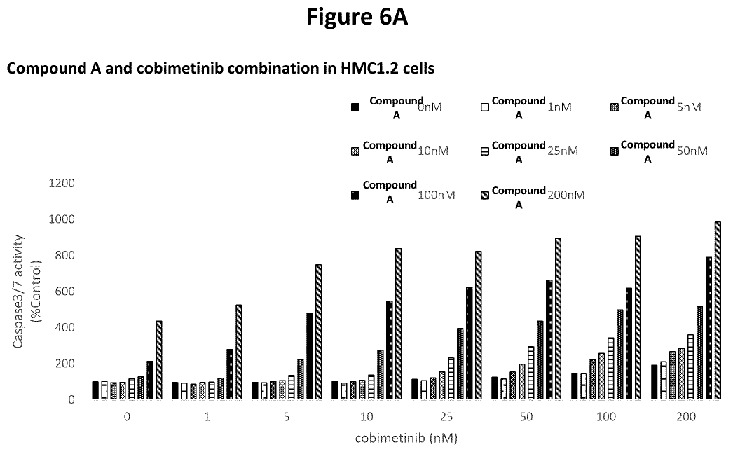

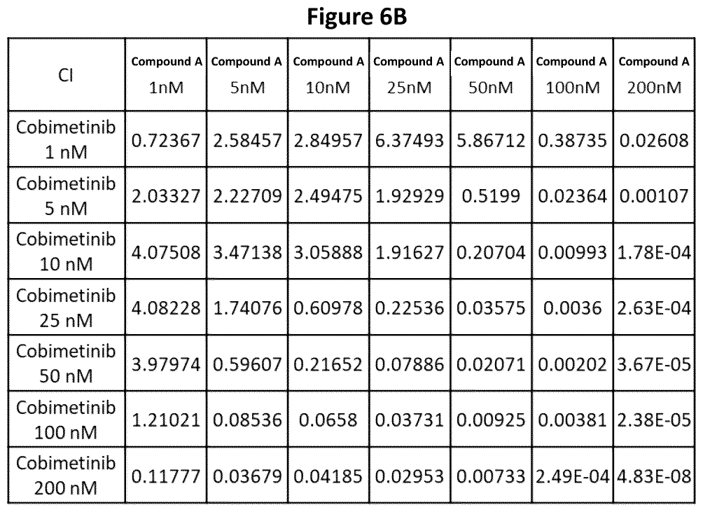

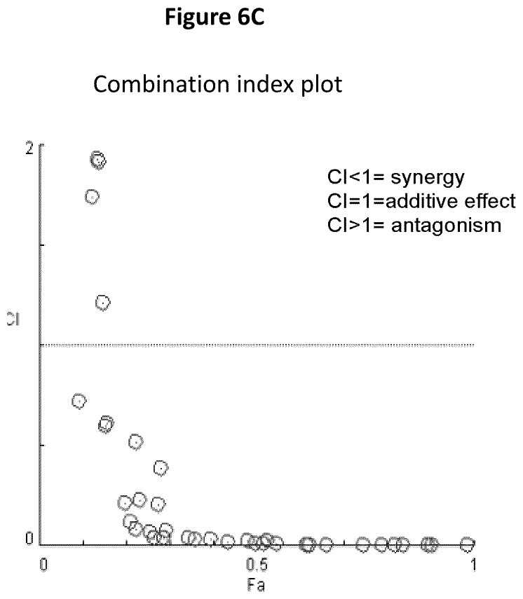

FIG. 6A is graphical representation showing the relative percentage of cell apoptosis determined for various treatments of Compound A and cobimetinib. FIG. 6B is a matrix for synergy chart based on the combination index (CI) method described by Chou and Talalay (1984) and the computer software of Chou and Martin (2005). Combination treatments with Compound A and the MEK inhibitor cobimetinib unexpectedly showed strong synergy for inducing apoptosis of HMC1.2 KIT V560G/D816V cells. FIG. 6C is a Combination Index Plot of the CI, demonstrating strong synergy for combination of Compound A with cobimetinib for inducing apoptosis of HMC1.2 KIT V560G/D816V mast cells.

### Example 7. Combination Treatment with Compound B and Cobimetinib Induces Apoptosis in Mastocytosis Cell Lines

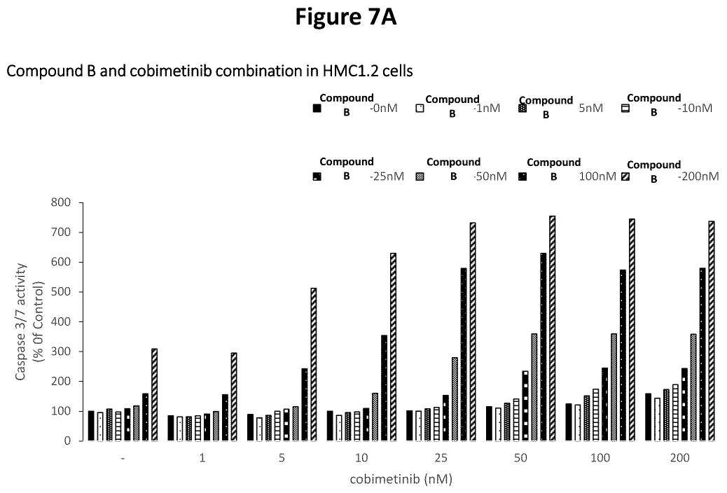

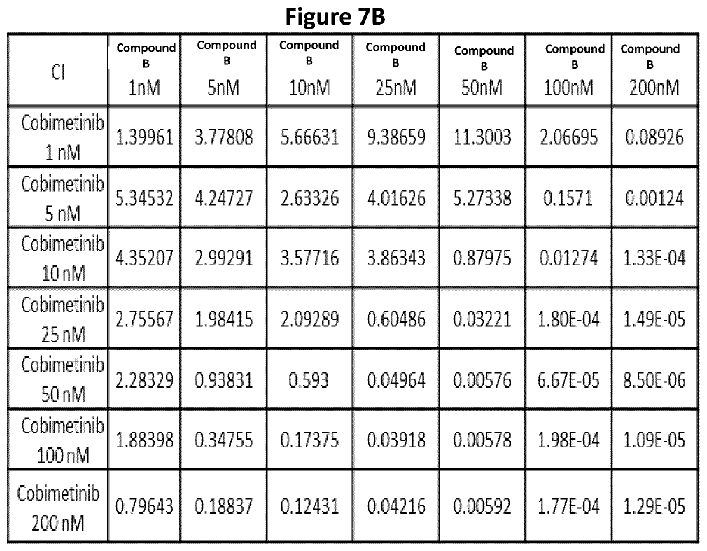

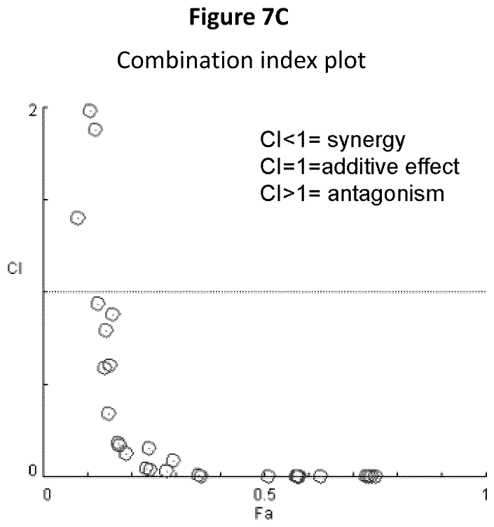

FIG. 7A is graphical representation showing the relative percentage of cell apoptosis determined for various treatments of Compound B and cobimetinib. FIG. 7B is a matrix for synergy chart based on the combination index (CI) method described by Chou and Talalay (1984) and the computer software of Chou and Martin (2005). CI<1 indicates synergism, CI=1 indicates additive effect, and CI>1 indicates antagonism. Combination treatments with Compound B and the MEK inhibitor cobimetinib unexpectedly showed strong synergy for inducing apoptosis of HMC1.2 KIT V560G/D816V cells. FIG. 7C is a Combination Index Plot of the CI, demonstrating strong synergy for combination of Compound B with cobimetinib for inducing apoptosis of HMC1.2 KIT V560G/D816V mast cells.

### Example 8. Combination Treatment with Compound A and Ulixertinib Induces Apoptosis in Mastocytosis Cell Lines

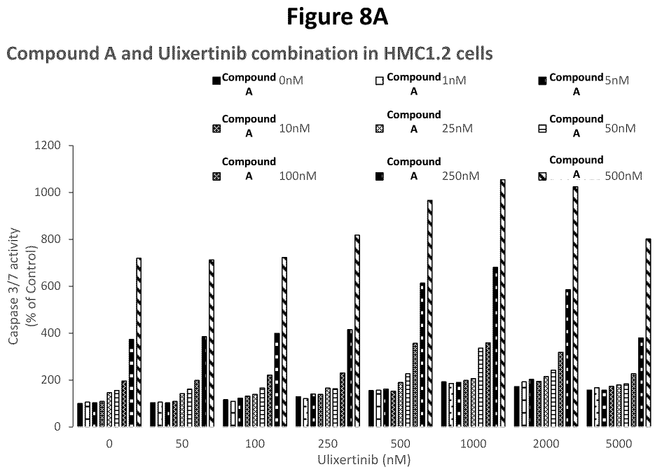

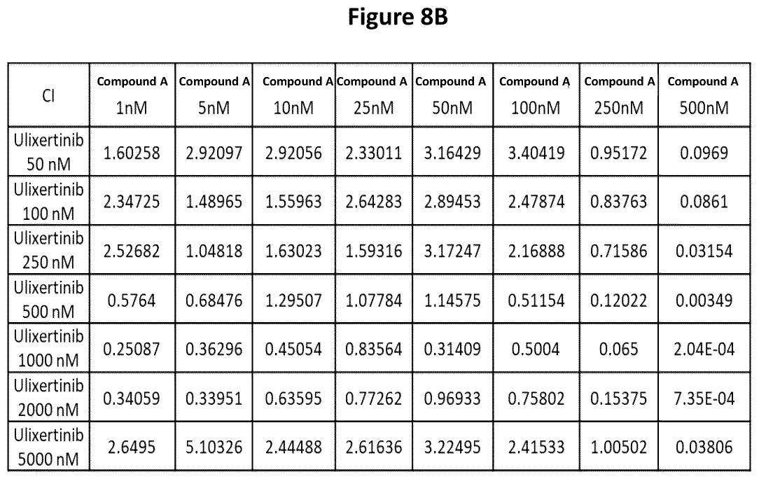

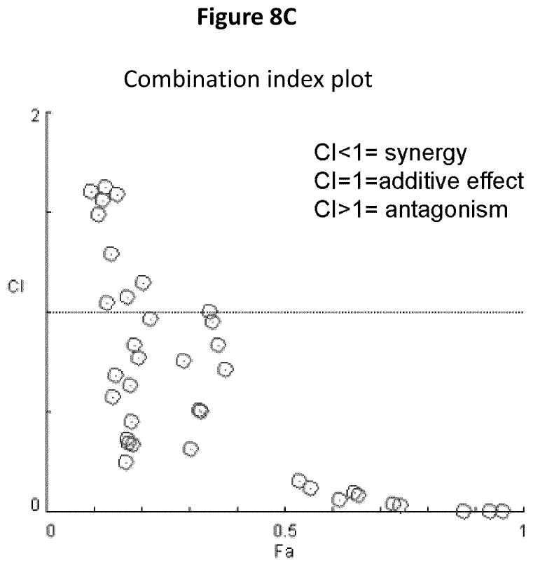

FIG. 8A is graphical representation showing the relative percentage of cell apoptosis determined for various treatments of Compound A and the ERK inhibitor ulixertinib. FIG. 8B is a matrix for synergy chart based on the combination index (CI) method described by Chou and Talalay (1984) and the computer software of Chou and Martin (2005). Combination treatments with Compound A and ulixertinib unexpectedly showed strong synergy for inducing apoptosis of HMC1.2 KIT V560G/D816V cells. FIG. 8C is a Combination Index Plot of the CI, demonstrating strong synergy for combination of Compound A with ulixertinib for inducing apoptosis of HMC1.2 KIT V560G/D816V mast cells.

### Example 9. Combination Treatment with Compound B and Ulixertinib Induces Apoptosis in Mastocytosis Cell Lines

Combination treatment of Compound B and the ERK inhibitor ulixertinib can be evaluated for induction of apoptosis in HMC1.2 KIT V560G/D816V mast cells. A matrix for synergy chart based on the combination index (CI) method can be generated as described by Chou and Talalay (1984) and the computer software of Chou and Martin (2005). Combination treatments with Compound B and ulixertinib can be used to show synergy for inducing apoptosis of HMC1.2 KIT V560G/D816V cells. A Combination Index Plot of the CI can be used to demonstrate synergy for combination of Compound B and ulixertinib for inducing apoptosis of HMC1.2 KIT V560G/D816V mast cells.

### Example 10. Combination Treatment with Compound A and SCH772984 Induces Apoptosis in Mastocytosis Cell Lines

Combination treatment of Compound A and the ERK inhibitor SCH772984 can be evaluated for induction of apoptosis in HMC1.2 KIT V560G/D816V mast cells. A matrix for synergy chart based on the combination index (CI) method can be generated as described by Chou and Talalay (1984) and the computer software of Chou and Martin (2005). Combination treatments with Compound A and SCH772984 can be used to show synergy for inducing apoptosis of HMC1.2 KIT V560G/D816V cells. A Combination Index Plot of the CI can be used to demonstrate synergy for combination of Compound A and SCH772984 for inducing apoptosis of HMC1.2 KIT V560G/D816V mast cells.

### Example 11. Combination Treatment with Compound B and SCH772984 Induces Apoptosis in Mastocytosis Cell Lines

Combination treatment of Compound B and the ERK inhibitor SCH772984 can be evaluated for induction of apoptosis in HMC1.2 KIT V560G/D816V mast cells. A matrix for synergy chart based on the combination index (CI) method can be generated as described by Chou and Talalay (1984) and the computer software of Chou and Martin (2005). Combination treatments with Compound A and SCH772984 can be used to show synergy for inducing apoptosis of HMC1.2 KIT V560G/D816V cells. A Combination Index Plot of the CI can be used to demonstrate synergy for combination of Compound B and SCH772984 for inducing apoptosis of HMC1.2 KIT V560G/D816V mast cells.

### Example 12. Combination Treatment with Compound A and LY3009120 Induces Apoptosis in Mastocytosis Cell Lines

Combination treatment of Compound A and the RAF inhibitor LY3009120 can be evaluated for induction of apoptosis in HMC1.2 KIT V560G/D816V mast cells. A matrix for synergy chart based on the combination index (CI) method can be generated as described by Chou and Talalay (1984) and the computer software of Chou and Martin (2005). Combination treatments with Compound A and LY3009120 can be used to show synergy for inducing apoptosis of HMC1.2 KIT V560G/D816V cells. A Combination Index Plot of the CI can be used to demonstrate strong synergy for combination of Compound A and LY3009120 for inducing apoptosis of HMC1.2 KIT V560G/D816V mast cells.

### Example 13. Combination Treatment with Compound B and LY3009120 Induces Apoptosis in Mastocytosis Cell Lines

Combination treatment of Compound B and the RAF inhibitor LY3009120 can be evaluated for induction of apoptosis in HMC1.2 KIT V560G/D816V mast cells. A matrix for synergy chart based on the combination index (CI) method can be generated as described by Chou and Talalay (1984) and the computer software of Chou and Martin (2005). Combination treatments with Compound B and LY3009120 can be used to show synergy for inducing apoptosis of HMC1.2 KIT V560G/D816V cells. A Combination Index Plot of the CI can be used to demonstrate synergy for combination of Compound B and LY3009120 for inducing apoptosis of HMC1.2 KIT V560G/D816V mast cells.

### Example 14. Combination Treatment with Compound A and Trametinib Leads to Synergistic Decrease in Colony Outgrowth of HMC1.2 KIT D816V Mast Cell Line

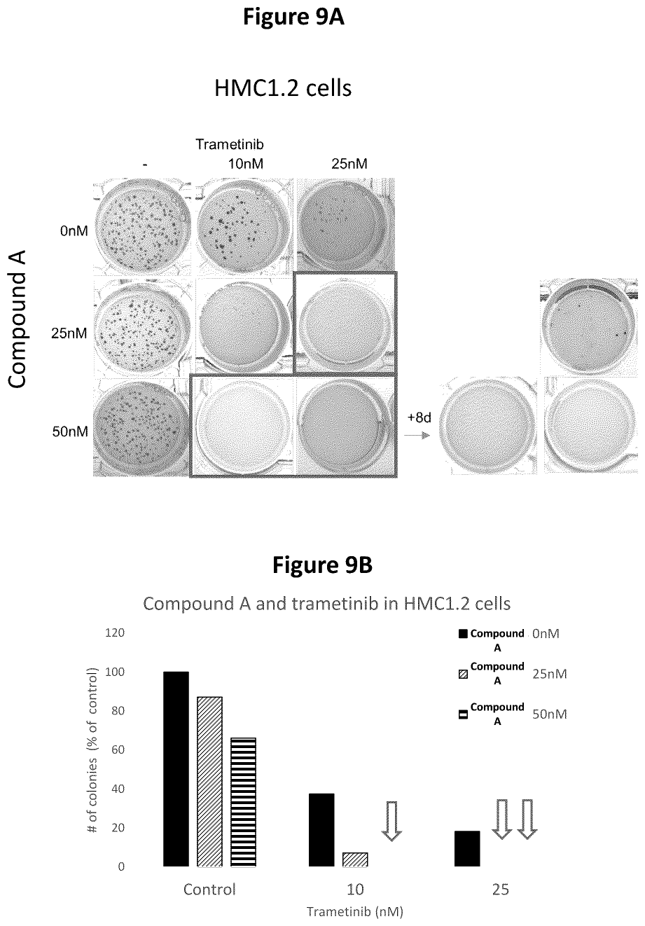

A study was performed to demonstrate that combination treatment with Compound A and the MEK inhibitor trametinib leads to decreased colony out growth of HMC1.2, compared to treatment with either single agent. 10,000 HMC1.2 cells were grown in soft agar and incubated with various concentrations of Compound A, trametinib, or a combination of Compound A and trametinib for 10 days. Drug treatments were removed and colony outgrowth of viable cells was monitored after 5 additional days. FIG. 9A is a representative picture of colony outgrowth of HMC1.2 mast cells after treatment with trametinib (0, 10, or 25 nM), Compound A (0, 25, or 50 nM) as single agents or as a matrix combination of various concentrations of Compound A with various concentrations of trametinib. Whereas the single agent treatment with Compound A or trametinib resulted in colony outgrowth at all concentrations 5 days after drug removal, the combination of Compound A with trametinib resulted in less colony outgrowth 5 days after drug removal. Combination of Compound A (25 nM) with trametinib (25 nM) resulted in a complete eradication of colony outgrowth after 5 days of drug removal. Combination of Compound A (50 nM) with trametinib (10 or 25 nM) unexpectedly resulted in complete eradication of outgrowth of viable HMC1.2 cells to the limit of detection as determined by visualization with 5× objective microscopy after 5 days of drug removal, whereas eradication was not observed upon treatment with single agent Compound A or single agent trametinib. Further extension of colony outgrowth to 8 additional days (a total of 13 days) after drug removal still maintained eradication to the limit of detection of colony outgrowth with the combination of Compound A (50 nM) and trametinib (10 and 25 nM). However, ˜15-20 colonies outgrew after extra 8 days of incubation with the combination of Compound A (25 nM) and trametinib (25 nM). (FIG. 9A right panel).

FIG. 9B is a graphical representation quantifying colony outgrowth of HMC1.2 mast cells after the various treatments from FIG. 9A. Combination treatment with Compound A at 50 nM and trametinib at either 10 nM or 25 nM unexpectedly resulted in eradication of colony outgrowth to the limit of detection as determined by visualization with 5× objective microscopy (see arrows, FIG. 9B). Combination treatment with Compound A at 25 nM and trametinib at 25 nM unexpectedly resulted in eradication of colony outgrowth to the limit of detection as determined by visualization with 5× objective microscopy (see arrows, FIG. 9B), Eradication was not observed upon treatment with either single agent Compound A or trametinib.

### Example 15. Combination Treatment with Compound B and Trametinib Leads to Synergistic Decrease in Colony Outgrowth of HMC1.2 KIT D816V Mast Cell Line

A study was also performed with Compound B and the MEK inhibitor trametinib to evaluate a decrease in colony outgrowth in HMC1.2 cells as described in Example 14.

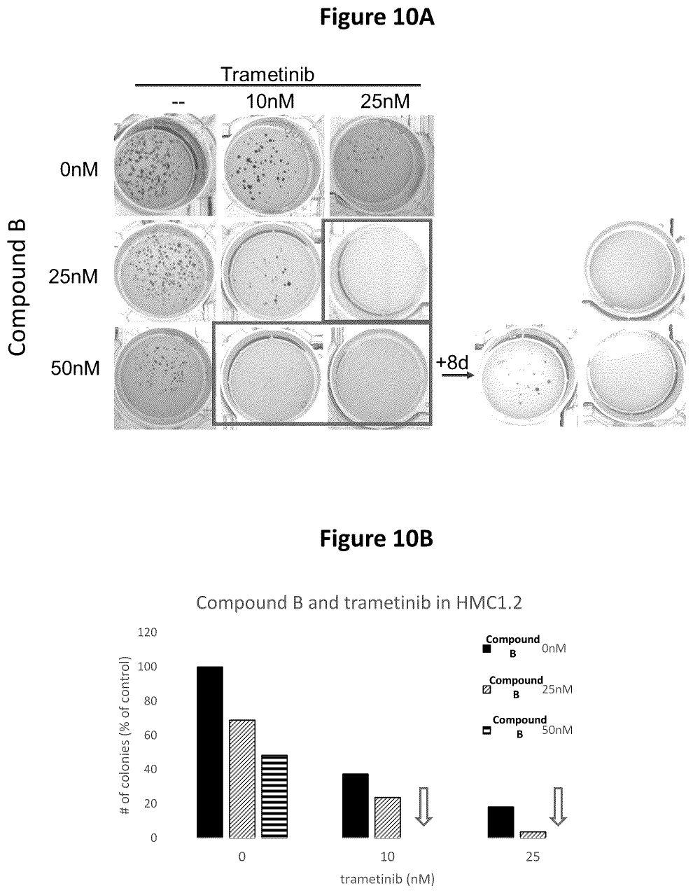

FIG. 10A is a representative picture of colony outgrowth of HMC1.2 mast cells after treatment with trametinib (0, 10, or 25 nM), Compound B (0, 25, or 50 nM) as single agents or as a matrix combination of various concentrations of Compound B with various concentrations of trametinib. Whereas the single agent treatment with Compound B or trametinib resulted in colony outgrowth at all concentrations 5 days after drug removal, the combination of Compound B with trametinib resulted in less colony outgrowth 5 days after drug removal. Combination of Compound B (25 nM) with trametinib (25 nM) resulted in a significant decrease in outgrowth of viable HMC1.2 cells after 5 days of drug removal. Combination of Compound B (50 nM) with trametinib (10 or 25 nM) unexpectedly resulted in complete eradication of outgrowth of viable HMC1.2 cells to the limit of detection as determined by visualization with 5× objective microscopy after 5 days of drug removal, whereas eradication was not observed upon treatment with single agent Compound B or single agent trametinib. Further extension of colony outgrowth to 8 additional days (a total of 13 days) after drug removal still maintained eradication to the limit of detection of colony outgrowth with the combination of Compound B (50 nM) and trametinib (25 nM). FIG. 10B is a graphical representation quantifying colony outgrowth of HMC1.2 mast cells after the various treatments from FIG. 10A. Combination treatment with Compound B at 50 nM and trametinib at either 10 nM or 25 nM unexpectedly resulted in eradication of colony outgrowth to the limit of detection as determined by visualization with 5× objective microscopy, whereas eradication was not observed upon treatment with either single agent Compound B or trametinib (see arrows, FIG. 10B).

### Example 16. Combination Treatment with Compound A and Binimetinib Leads to Synergistic Decrease in Colony Outgrowth of HMC1.2 KIT D816V Mast Cell Line

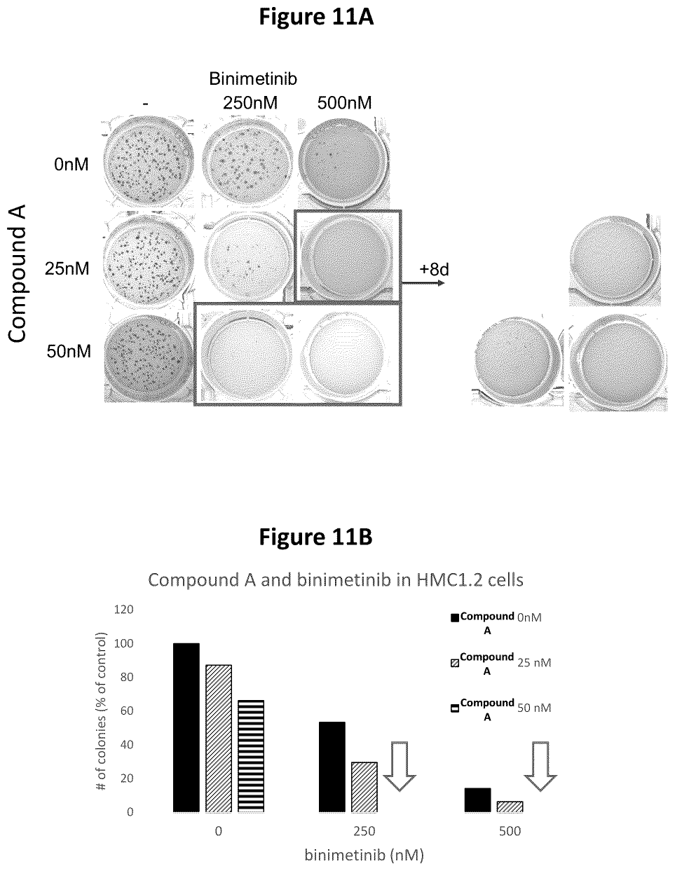

FIG. 11A is a representative picture of colony outgrowth of HMC1.2 mast cells after treatment with binimetinib (0, 250, or 500 nM), Compound A (0, 25, or 50 nM) as single agents or as a matrix combination of various concentrations of Compound A with various concentrations of binimetinib. Whereas the single agent treatment with Compound A or binimetinib resulted in colony outgrowth at all concentrations 5 days after drug removal, the combination of Compound A with binimetinib resulted in less colony outgrowth 5 days after drug removal. Combination of Compound A (25 nM) with binimetinib (250 nM) resulted in a significant decrease in outgrowth of viable HMC1.2 cells after 5 days of drug removal. Combination of Compound A (50 nM) with binimetinib (250 nM or 500 nM) unexpectedly resulted in complete eradication of outgrowth of viable HMC1.2 cells to the limit of detection as determined by visualization with 5× objective microscopy after 5 days of drug removal, whereas eradication was not observed upon treatment with single agent Compound A or single agent binimetinib. Further extension of colony outgrowth to 8 additional days (a total of 13 days) after drug removal still maintained eradication to the limit of detection of colony outgrowth with the combination of Compound A (50 nM) and binimetinib (500 nM). However, ˜10-15 colonies outgrew with the combination of Compound A (50 nM) and binimetinib (250 nM).

FIG. 11B is a graphical representation quantifying colony outgrowth of HMC1.2 mast cells after the various treatments from FIG. 11A. Combination treatment with Compound A at 50 nM and binimetinib at 250 nM or 500 nM unexpectedly resulted in eradication of colony outgrowth to the limit of detection as determined by visualization with 5× objective microscopy, whereas eradication was not observed upon treatment with either single agent Compound A or binimetinib (see arrows, FIG. 11B).

### Example 17. Combination Treatment with Compound B and Binimetinib Leads to Synergistic Decrease in Colony Outgrowth of HMC1.2 KIT D816V Mast Cell Line

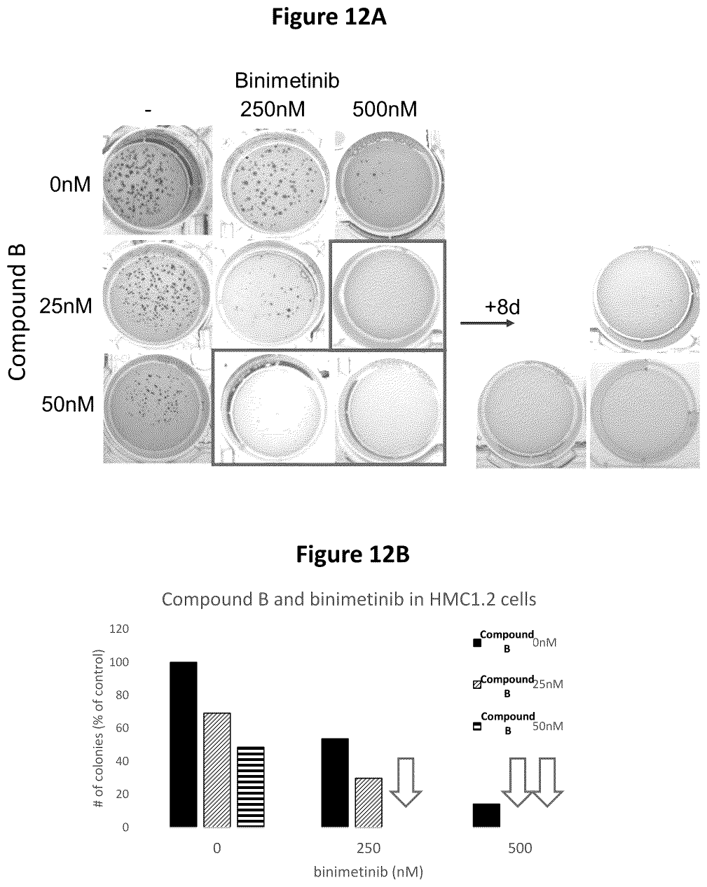

FIG. 12A is a representative picture of colony outgrowth of HMC1.2 mast cells after treatment with binimetinib (0, 250, or 500 nM), Compound B (0, 25, or 50 nM) as single agents or as a matrix combination of various concentrations of Compound B with various concentrations of binimetinib. Whereas the single agent treatment with Compound B or binimetinib resulted in colony outgrowth at all concentrations 5 days after drug removal, the combination of Compound B with binimetinib resulted in less colony outgrowth 5 days after drug removal. Combination of Compound B (25 nM) with binimetinib (250 nM) resulted in a significant decrease in outgrowth of viable HMC1.2 cells after 5 days of drug removal. Combination of Compound B (25 nM) with binimetinib (500 nM) resulted in complete eradication of outgrowth of viable HMC1.2 cells after 5 days of drug removal to the limit of detection as determined by visualization with 5× objective microscopy. Combination of Compound B (50 nM) with binimetinib (250 nM or 500 nM) unexpectedly resulted in complete eradication of outgrowth of viable HMC1.2 cells to the limit of detection as determined by visualization with 5× objective microscopy after 5 days of drug removal, whereas eradication was not observed upon treatment with single agent Compound B or single agent binimetinib. Further extension of colony outgrowth to 8 additional days (a total of 13 days) after drug removal still maintained eradication to the limit of detection of colony outgrowth with the combination of Compound B (50 nM) and binimetinib (250 or 500 nM) and Compound B (25 nM) and binimetinib (500 nM).

FIG. 12B is a graphical representation quantifying colony outgrowth of HMC1.2 mast cells after the various treatments from FIG. 12A. Combination treatment with Compound B at 25 nM and binimetinib at 500 nM and combination treatment with Compound B at 50 nM and binimetinib at either 250 nM or 500 nM unexpectedly resulted in eradication of colony outgrowth to the limit of detection as determined by visualization with 5× objective microscopy, whereas eradication was not observed upon treatment with either single agent Compound B or binimetinib (see arrows, FIG. 12B).

### Example 18. Combination Treatment with Compound A and Cobimetinib Leads to Synergistic Decrease in Colony Outgrowth of HMC1.2 KIT D816V Mast Cell Line

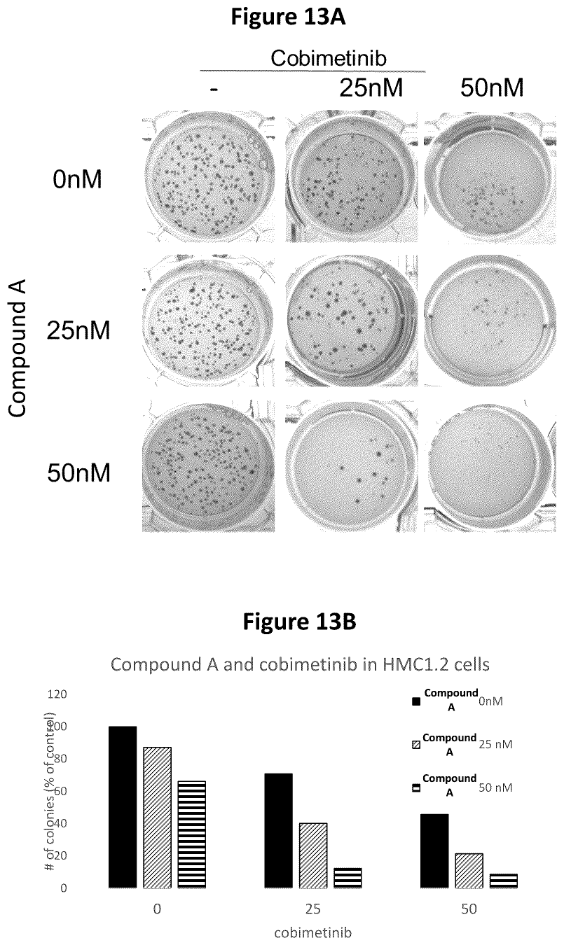

FIG. 13A is a representative picture of colony outgrowth of HMC1.2 mast cells after treatment with cobimetinib (0, 25, or 50 nM), Compound A (0, 25, or 50 nM) as single agents or as a matrix combination of various concentrations of Compound A with various concentrations of cobimetinib. Whereas the single agent treatment with Compound A or cobimetinib resulted in colony outgrowth at all concentrations 5 days after drug removal, the combination of Compound A with cobimetinib resulted in less colony outgrowth 5 days after drug removal compared to either single agent treatment. Combination of Compound A (50 nM) with cobimetinib (25 or 50 nM) resulted in a significant decrease in outgrowth of viable HMC1.2 cells after 5 days of drug removal.

FIG. 13B is a graphical representation quantifying colony outgrowth of HMC1.2 mast cells after the various treatments from FIG. 13A. Combination treatment with Compound A at 50 nM and cobimetinib at either 25 nM or 50 nM resulted in significant decrease of colony outgrowth compared to either single agent Compound A or cobimetinib.

### Example 19. Combination Treatment with Compound B and Cobimetinib Leads to Synergistic Decrease in Colony Outgrowth of HMC1.2 KIT D816V Mast Cell Line

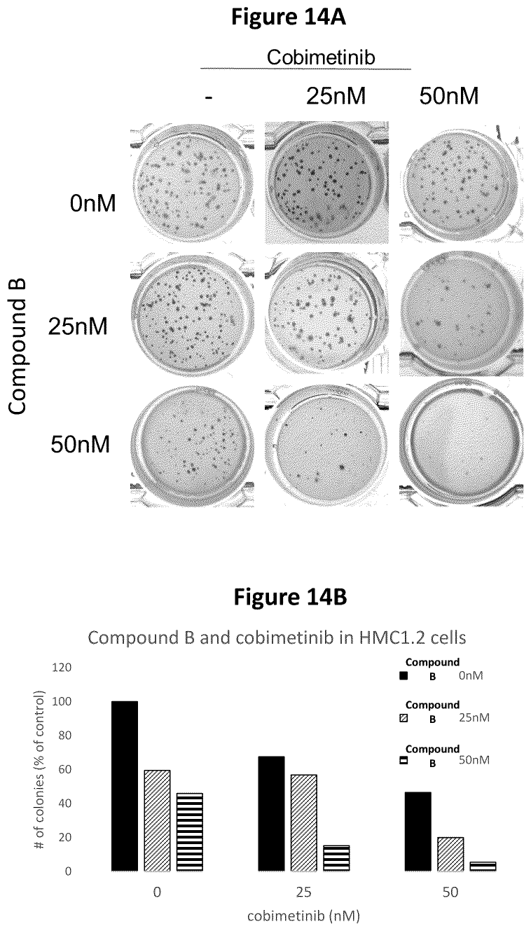

FIG. 14A is a representative picture of colony outgrowth of HMC1.2 mast cells after treatment with cobimetinib (0, 25, or 50 nM), Compound B (0, 25, or 50 nM) as single agents or as a matrix combination of various concentrations of Compound B with various concentrations of cobimetinib. Whereas the single agent treatment with Compound B or cobimetinib resulted in colony outgrowth at all concentrations 5 days after drug removal, the combination of Compound B with cobimetinib resulted in less colony outgrowth 5 days after drug removal compared to either single agent treatment. Combination of Compound B (50 nM) with cobimetinib (25 or 50 nM) resulted in a significant decrease in outgrowth of viable HMC1.2 cells after 5 days of drug removal.

FIG. 14B is a graphical representation quantifying colony outgrowth of HMC1.2 mast cells after the various treatments from FIG. 14A. Combination treatment with Compound B at 50 nM and cobimetinib at either 25 nM or 50 nM resulted in significant decrease of colony outgrowth compared to either single agent Compound B or cobimetinib.

### Example 20. Combination Treatment with Compound A and the ERK Inhibitor Ulixertinib Leads to a Decrease in Colony Outgrowth of HMC1.2 KIT D816V Mast Cell Line

Combination treatment of Compound A and ulixertinib can be evaluated for inhibition of colony outgrowth of HMC1.2 KIT D816V mast cell line according to the method of Example 14.

### Example 21. Combination Treatment with Compound B and the ERK Inhibitor Ulixertinib Leads to a Decrease in Colony Outgrowth of HMC1.2 KIT D816V Mast Cell Line

Combination treatment of Compound B and ulixertinib can be evaluated for inhibition of colony outgrowth of HMC1.2 KIT D816V mast cell line according to the method of Example 14.

### Example 22. Combination Treatment with Compound A and the RAF Inhibitor LY3009120 Leads to a Decrease in Colony Outgrowth of HMC1.2 KIT D816V Mast Cell Line

Combination treatment of Compound A and the RAF inhibitor LY3009120 can be evaluated for inhibition of colony outgrowth of HMC1.2 KIT D816V mast cell line according to the method of Example 14.

### Example 23. Combination Treatment with Compound B and LY3009120 Leads to a Decrease in Colony Outgrowth of HMC1.2 KIT D816V Mast Cell Line

Combination treatment of Compound B and the RAF inhibitor LY3009120 can be evaluated for inhibition of colony outgrowth of HMC1.2 KIT D816V mast cell line according to the method of Example 14.

### Example 24. Combination Treatment with Compound A and the MEK Inhibitor Trametinib Induces Apoptosis in Mutant N-ras G12D Transfected HMC1.2 KIT V560G/D816V Mastocytosis Cell Line

A study was performed to demonstrate that combination treatment with Compound A and trametinib induced apoptosis in mutant N-ras G12D transfected HMC1.2 V560G/D816V cells. Assays were conducted in 96 well plates with 10,000 cells seeded per well. Cells were treated with vehicle control, Compound A, trametinib, or combinations thereof at varying concentrations, and the cells were allowed to grow for 24 and 48 hours. Apoptosis was assessed by measuring caspase 3/7 activity.

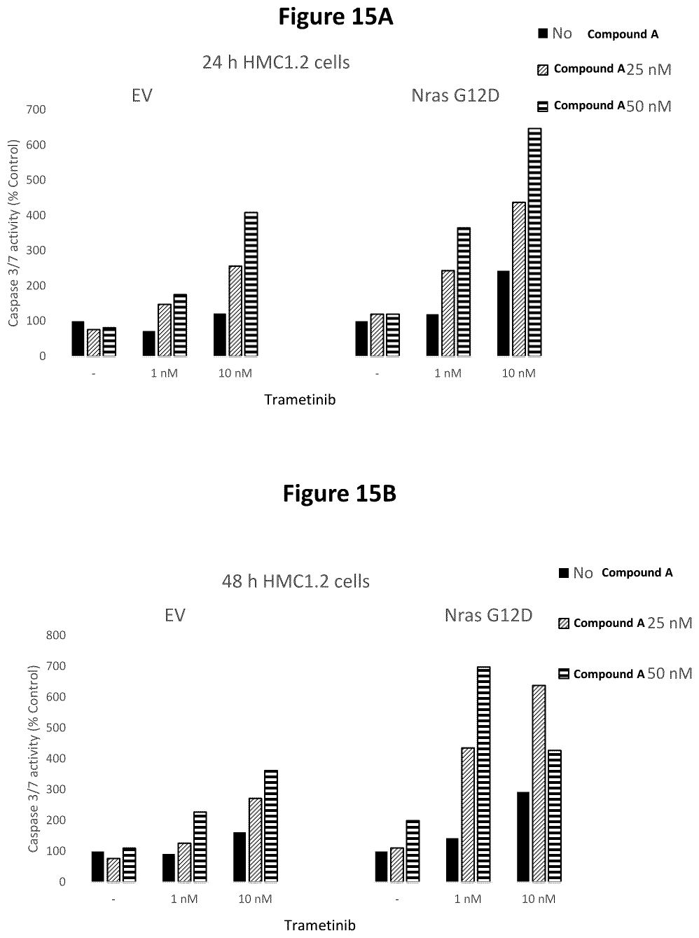

FIG. 15A is a graphical representation showing the relative percentage of caspase activity determined for various treatments at 24 hours. Combination treatment with Compound A and trametinib for 24 hours induced apoptosis of empty vector (EV)-transfected (left panel) HMC1.2 V560G/D816V cells or mutant N-ras G12D transfected (right panel) HMC1.2 V560G/D816V cells. Combination of Compound A with trametinib led to a synergistic increase in apoptosis in EV-transfected and in N-ras G12D transfected HMC1.2 cells.

FIG. 15B is a graphical representation showing the relative percentage of caspase activity determined for various treatments at 48 hours. Combination treatment with Compound A and trametinib for 48 hours induced apoptosis of empty vector (EV)-transfected (left panel) HMC1.2 V560G/D816V cells or mutant N-ras G12D transfected (right panel) HMC1.2 V560G/D816V cells. Combination of Compound A with trametinib led to a synergistic increase in apoptosis in EV-transfected and in N-ras G12D transfected HMC1.2 cells. Apoptosis was higher in N-ras transfected cells than EV transfected cells.

### Example 25. Combination Treatment with Compound A and the MEK Inhibitor Cobimetinib Induces Apoptosis in Mutant N-ras G12D Transfected HMC1.2 KIT V560G/D816V Mastocytosis Cell Line

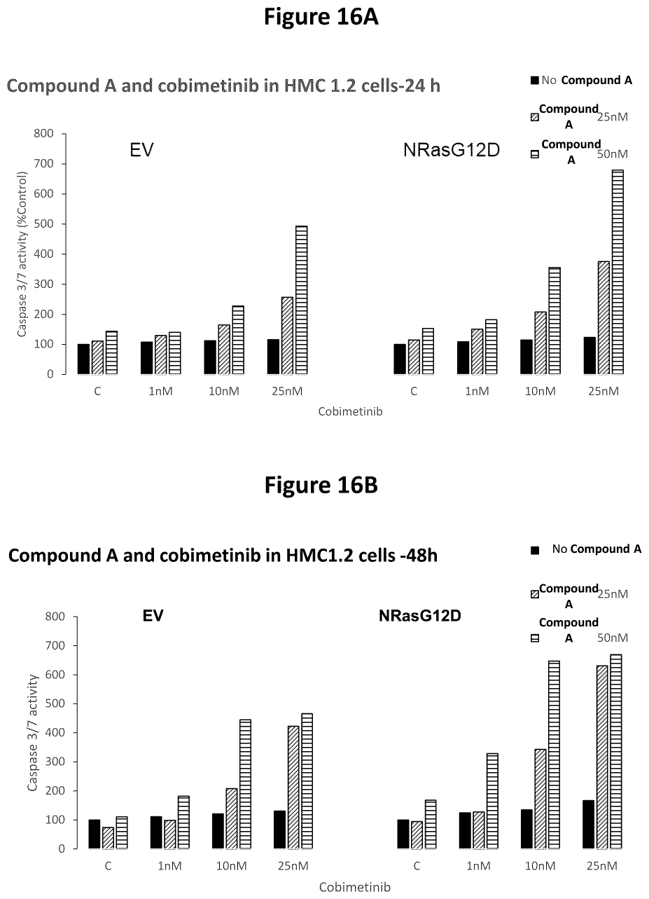

FIG. 16A is a graphical representation showing the relative percentage of caspase activity determined for various treatments at 24 hours. Combination treatment with Compound A and cobimetinib for 24 hours induced apoptosis of empty vector (EV)-transfected (left panel) HMC1.2 V560G/D816V cells or mutant N-ras G12D transfected (right panel) HMC1.2 V560G/D816V cells. Combination of Compound A with cobimetinib led to a synergistic increase in apoptosis in EV-transfected and in N-ras G12D transfected HMC1.2 cells. Apoptosis was higher in N-ras transfected cells than in EV transfected cells.

FIG. 16B is a graphical representation showing the relative percentage of caspase activity determined for various treatments at 48 hours. Combination treatment with Compound A and cobimetinib for 48 hours induced apoptosis of empty vector (EV)-transfected (left panel) HMC1.2 V560G/D816V cells or mutant N-ras G12D transfected (right panel) HMC1.2 V560G/D816V cells. Combination of Compound A with cobimetinib led to a synergistic increase in apoptosis in EV-transfected and in N-ras G12D transfected HMC1.2 cells. Apoptosis was higher in N-ras transfected cells than in EV transfected cells.

### Example 26. Combination Treatment with Compound A and the MEK Inhibitor Trametinib Leads to Synergistic Decrease in Colony Outgrowth of HMC1.2 KIT D816V Mast Cell Line Transfected with N-ras G12D or Empty Vector

A study was performed to demonstrate that combination treatment with Compound A and trametinib leads to decreased colony out growth of HMC1.2 transfected with empty vector (EV) or transfected with N-ras G12D, compared to treatment with either single agent. HMC1.2 cells were incubated with various concentrations of Compound A, trametinib, or a combination of Compound A and trametinib for 10 days. Drug treatments were removed and colony outgrowth of viable cells was monitored after 5 or 13 additional days.

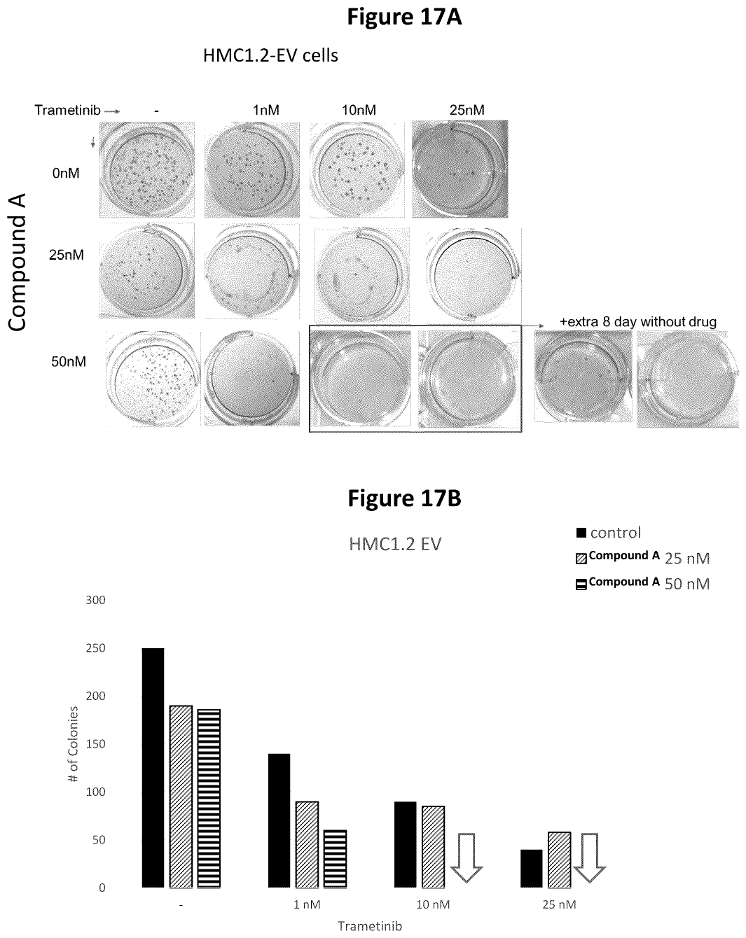

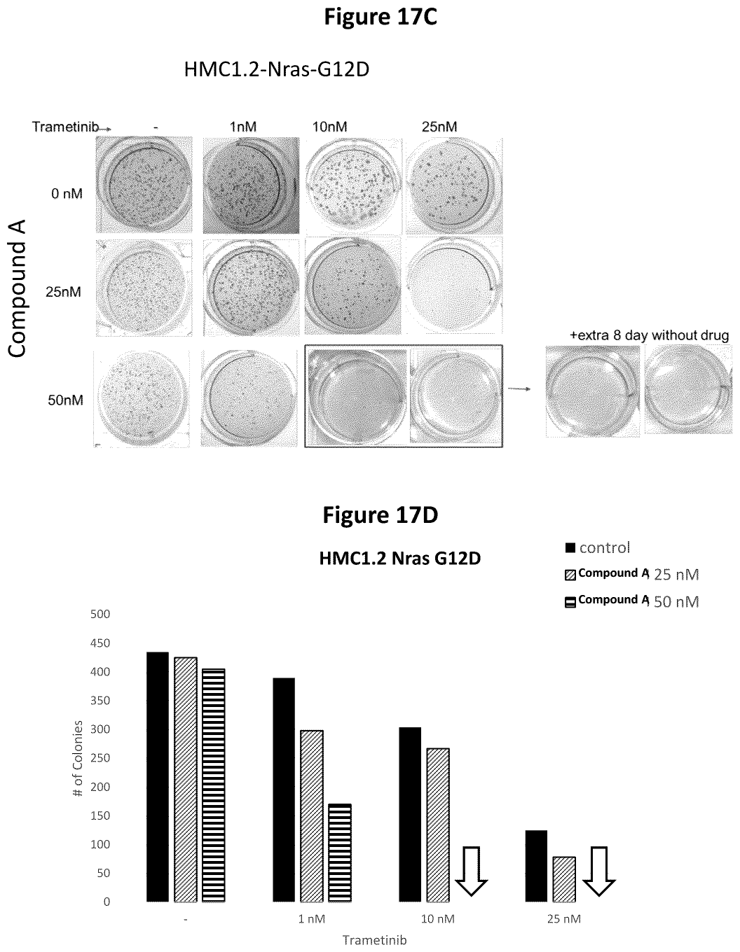

FIG. 17A is a representative picture of colony outgrowth of EV-transfected HMC1.2 mast cells after treatment with trametinib (0, 1, 10, or 25 nM), Compound A (0, 25, or 50 nM) as single agents or as a matrix combination of various concentrations of Compound A with various concentrations of trametinib. Whereas the single agent treatment with Compound A or trametinib resulted in colony outgrowth at all concentrations 5 days after drug removal, the combination of Compound A with trametinib resulted in less colony outgrowth 5 days after drug removal. Combination of Compound A (50 nM) with trametinib (1 nM) resulted in a significant reduction in colony outgrowth in combination with 1 nM trametinib compared to single agent Compound A or single agent trametinb. Combination of Compound A (50 nM) with trametinib (10 or 25 nM) unexpectedly resulted in complete eradication of outgrowth of viable HMC1.2 cells to the limit of detection as determined by visualization with 5× objective microscopy after 5 days of drug removal, whereas eradication was not observed upon treatment with single agent Compound A or single agent trametinib.

Further extension of colony outgrowth for 8 additional days after drug removal (total of 13 days after drug removal) still maintained eradication to the limit of detection of colony outgrowth with the combination of Compound A (50 nM) and trametinib (25 nM).

FIG. 17B is a graphical representation quantifying colony outgrowth of EV-transfected HMC1.2 mast cells after the various treatments from FIG. 17A. Combination treatment with Compound A at 50 nM and trametinib at either 10 nM or 25 nM unexpectedly resulted in eradication of colony outgrowth to the limit of detection as determined by visualization with 5× objective microscopy, whereas eradication was not observed upon treatment with either single agent Compound A or trametinib (see arrows, FIG. 17B).

FIG. 17C is a representative picture of colony outgrowth of N-ras G12D-transfected HMC1.2 cells after treatment with trametinib (0, 1, 10, or 25 nM), Compound A (0, 25, or 50 nM) as single agents or as a matrix combination of various concentrations of Compound A with various concentrations of trametinib. Whereas the single agent treatment with Compound A or trametinb resulted in colony outgrowth at all concentrations 5 days after drug removal, the combination of Compound A with trametinib resulted in less colony outgrowth 5 days after drug removal. Combination of Compound A (25 nM) with trametinib (25 nM) resulted in a significant decrease in outgrowth of viable HMC1.2 cells after 5 days of drug removal, and combination of Compound A (50 nM) with trametinib (10 or 25 nM) unexpectedly resulted in complete eradication of outgrowth of viable N-ras G12D transfected HMC1.2 cells to the limit of detection as determined by visualization with 5× objective microscopy after 5 days of drug removal, whereas eradication was not observed upon treatment with either single agent Compound A or trametinib. Further extension of colony outgrowth by 8 additional days after drug removal (total of 13 days after drug removal) still maintained eradication of colony outgrowth with the combination of Compound A (50 nM) and trametinib (25 nM) (FIG. 17C).

FIG. 17D is a graphical representation quantifying colony outgrowth of the various treatments from FIG. 17C. Combination treatments with Compound A and trametinib resulted in superior blockade of colony outgrowth compared to treatment with either single agent. Combination treatment with Compound A at 50 nM and trametinib at either 10 nM or 25 nM unexpectedly resulted in eradication of colony outgrowth to the limit of detection as determined by visualization with 5× objective microscopy after 5 days of drug removal, whereas eradication was not observed upon treatment with either single agent Compound A or trametinib (see arrows, FIG. 17D).

### Example 27. Combination Treatment with Compound A and Cobimetinib Leads to Synergistic Decrease in Colony Outgrowth of HMC1.2 KIT D816V Mast Cell Line Transfected with N-ras G12D or Empty Vector

A study was performed to demonstrate that combination treatment with Compound A and cobimetinib leads to decreased colony out growth of HMC1.2 transfected with empty vector (EV) or transfected with N-ras G12D, compared to treatment with either single agent. HMC1.2 cells were incubated with various concentrations of Compound A, cobimetinib, or a combination of Compound A and cobimetinib for 10 days. Drug treatments were removed and colony outgrowth of viable cells was monitored after 5 or 10 additional days.

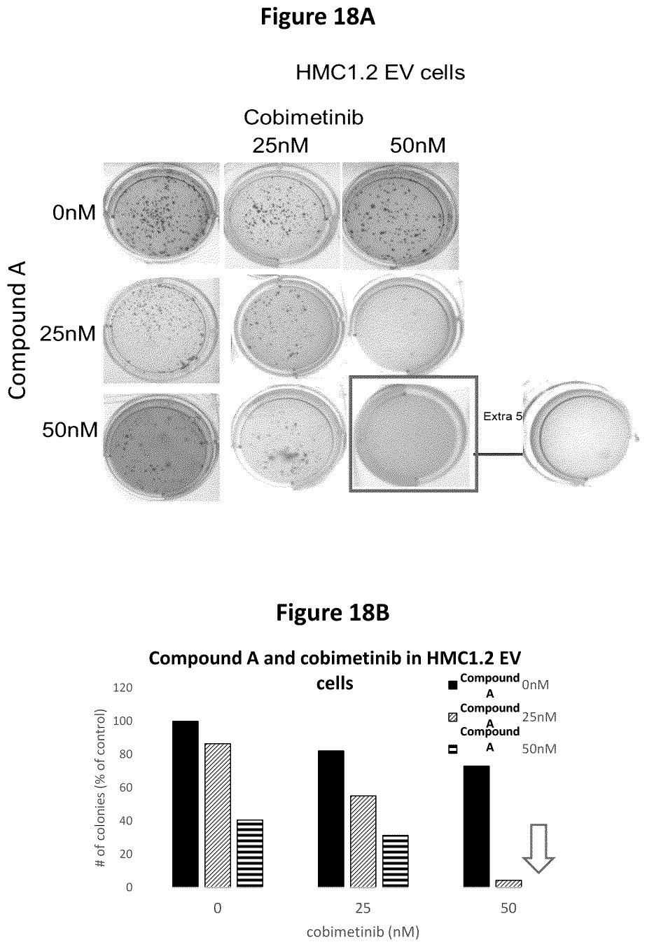

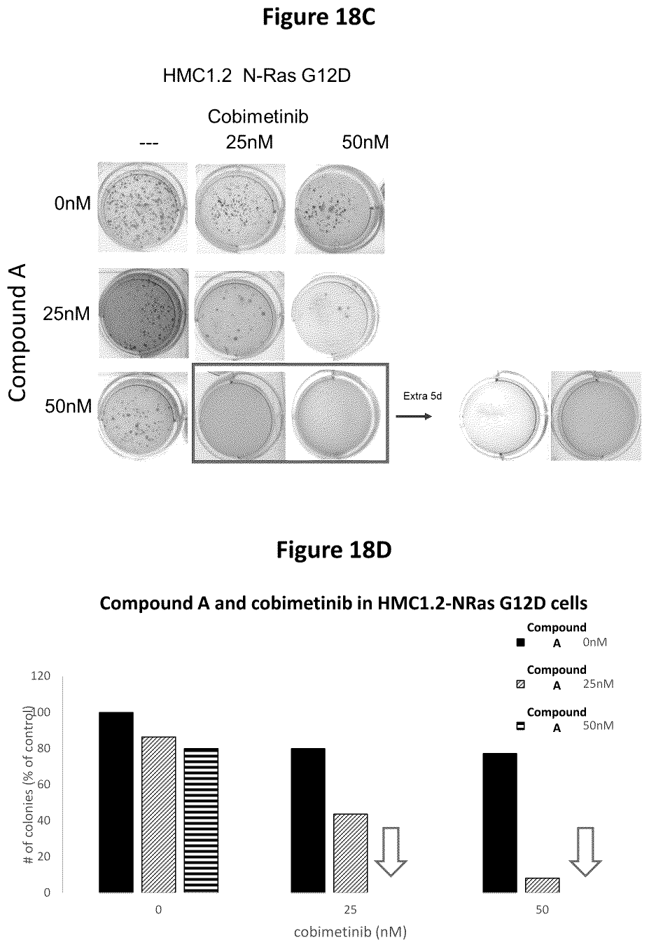

FIG. 18A is a representative picture of colony outgrowth of EV-transfected HMC1.2 mast cells after treatment with cobimetinib (25 or 50 nM), Compound A (0, 25, or 50 nM) as single agents or as a matrix combination of various concentrations of Compound A with various concentrations of cobimetinib. Whereas the single agent treatment with Compound A or cobimetinib resulted in colony outgrowth at all concentrations 5 days after drug removal, the combination of Compound A with cobimetinib resulted in less colony outgrowth 5 days after drug treatment removal. Combination of Compound A (25 nM) with cobimetinib (25 and 50 nM) resulted in a significant decrease in outgrowth of viable HMC1.2 cells after 5 days of drug treatment removal. Combination of Compound A (50 nM) with cobimetinib (50 nM) unexpectedly resulted in complete eradication of outgrowth of viable HMC1.2 cells to the limit of detection as determined by visualization with 5× objective microscopy after 5 days of drug treatment removal, whereas eradication was not observed upon treatment with single agent Compound A or single agent cobimetinib. Further extension of colony outgrowth by 5 additional days after drug treatment removal (total of 10 days after drug removal) still maintained eradication to the limit of detection of colony outgrowth with the combination of Compound A (50 nM) and cobimetinib (50 nM).

FIG. 18B is a graphical representation quantifying colony outgrowth of EV-transfected HMC1.2 mast cells after the various treatments from FIG. 18A. Combination treatment with Compound A at 50 nM and cobimetinib at 50 nM unexpectedly resulted in eradication of colony outgrowth to the limit of detection as determined by visualization with 5× objective microscopy, whereas eradication was not observed upon treatment with either single agent Compound A or cobimetinib (see arrows, FIG. 18B).

FIG. 18C is a representative picture of colony outgrowth of N-ras G12D-transfected HMC1.2 mast cells after treatment with cobimetinib (0, 25 or 50 nM), Compound A (0, 25, or 50 nM) as single agents or as a matrix combination of various concentrations of Compound A with various concentrations of cobimetinib. Whereas the single agent treatment with Compound A or cobimetinib resulted in colony outgrowth at all concentrations 5 days after drug treatment removal, the combination of Compound A with cobimetinib resulted in less colony outgrowth 5 days after drug treatment removal. Combination of Compound A (25 nM) with cobimetinib (25 and 50 nM) resulted in a significant decrease in outgrowth of viable HMC1.2 cells after 5 days of drug removal. Combination of Compound A (50 nM) with cobimetinib (25 or 50 nM) unexpectedly resulted in complete eradication of outgrowth of viable HMC1.2 cells to the limit of detection as determined by visualization with 5× objective microscopy after 5 days of drug treatment removal, whereas eradication was not observed upon treatment with single agent Compound A or single agent cobimetinib. Further extension of colony outgrowth to 10 additional days after drug treatment removal still maintained eradication to the limit of detection of colony outgrowth with the combination of Compound A (50 nM) and cobimetinib (25 or 50 nM).

FIG. 18D is a graphical representation quantifying colony outgrowth of the various treatments from FIG. 18C. Combination treatments with Compound A and cobimetinib resulted in superior blockade of colony outgrowth compared to treatment with either single agent. Combination treatment with Compound A at 50 nM and cobimetinib at 25 or 50 nM unexpectedly resulted in eradication of colony outgrowth to the limit of detection as determined by visualization with 5× objective microscopy after 5 days of drug removal, whereas eradication was not observed upon treatment with either single agent Compound A or cobimetinib (see arrows, FIG. 18D).

### EQUIVALENTS

Those skilled in the art will recognize, or be able to ascertain, using no more than routine experimentation, numerous equivalents to the specific embodiments described specifically in this disclosure. Such equivalents are intended to be encompassed in the scope of the following claims.

1. A method of treating mastocytosis in a patient in need thereof, comprising administering to the patient:
an effective amount of a c-KIT inhibitor, wherein the c-KIT inhibitor is 1-(5-(7-amino-1-ethyl-2-oxo-1,2-dihydro-1,6-naphthyridin-3-yl)-4-bromo-2-fluorophenyl)-3-phenylurea, or a pharmaceutically acceptable salt thereof; and
an effective amount of one or more MAPKAP pathway inhibitors selected from the group consisting of trametinib, cobimetinib, binimetinib, and ulixertinib.

2. (canceled)

3. The method of claim 1, wherein the mastocytosis has a c-KIT mutation.

4. The method of claim 3, wherein the c-KIT mutation is an activating mutation.

5. The method of claim 1, wherein the mastocytosis comprises mast cells having a primary mutation in exon 17 of a c-KIT gene.

6. (canceled)

7. The method of claim 5, wherein the primary mutation is one of D816V, D816Y, D816F, D816H, F522C, K5091, V560G, V559G, and del419.

8-15. (canceled)

16. The method of claim 1, wherein the mastocytosis is systemic mastocytosis.

17. The method of claim 16, wherein the systemic mastocytosis is selected from the group consisting of indolent systemic mastocytosis, systemic smoldering mastocytosis, systemic mastocytosis with associated clonal hematological non-mast cell lineage disease, aggressive systemic mastocytosis, mast cell leukemia, and mast cell sarcoma.

18-22. (canceled)

23. The method of claim 1, wherein the mastocytosis is cutaneous mastocytosis.

24. The method of claim 23, wherein the mastocytosis is selected from the group consisting of: maculopapular cutaneous mastocytosis, mastocytoma, and diffuse cutaneous mastocytosis.

25-29. (canceled)

30. The method of claim 1, wherein the c-KIT inhibitor and the MAPKAP pathway inhibitor are administered substantially concurrently or sequentially.

31. The method of claim 1, further comprising administering another cancer-targeted therapeutic agent, cancer-targeted biological, immune checkpoint inhibitor, or chemotherapeutic agent.

32. (canceled)

33. A method of treating a systemic mastocytosis in a patient in need thereof, comprising administering to the patient:
an effective amount of 1-(5-(7-amino-1-ethyl-2-oxo-1,2-dihydro-1,6-naphthyridin-3-yl)-4-bromo-2-fluorophenyl)-3-phenylurea, or a pharmaceutically acceptable salt thereof; and an effective amount of one or more MAPKAP pathway inhibitors selected from the group consisting of trametinib, cobimetinib, binimetinib, and ulixertinib.

34-47. (canceled)
